(19) United States
(12) Patent Application Publication
(10) Pub. No.: US 2008/0003250 A1
(43) Pub. Date: Jan. 3, 2008

(54) NOVEL THERAPY FOR HERPESVIRUS INFECTION

(76) Inventors: BARRY JOSEPH MARGULIES, Lutherville, MD (US); Tory Patricia Johnson, Crofton, MD (US)

Correspondence Address: MOORE & VAN ALLEN PLLC, P.O. BOX 13706, Research Triangle Park, NC, 27709, US

(21) Appl. No.: 11/766,298

(22) Filed: Jun. 21, 2007

Related U.S. Application Data
(60) Provisional application No. 60/805,381, filed on Jun. 21, 2006.

(51) Int. Cl. A61K 31/522; A61K 9/00; A61P 31/22

(52) U.S. Cl. 424/422; 514/263.38

(57) ABSTRACT

The present invention relates to an implantable drug delivery device comprising a polymeric or polymeric containing material in combination with a therapeutic compound effective for the treatment of a member of the herpesvirus family, wherein the therapeutic compound is in an amount that will effectively treat HSV-1, HSV 2 and/or VZV or reduce reactivation, and wherein the implantable drug delivery device is positioned at or near the site of latent infection or at the site of observed clinical symptoms. The therapeutic compound may include any active antiherpes agent including acyclovir, guanosine, valacyclovir or functionally active analogues.

### CROSS-REFERENCE TO RELATED APPLICATION

The present application claims priority to U.S. Provisional Patent Application No. 60/805,381 filed on Jun. 21, 2006, the contents of which are hereby incorporated by reference herein.

### BACKGROUND THE INVENTION

1. Field of the Invention

The present invention relates to treatment methods for Human herpes simplex virus (HSV), and more specifically, to an implant system for the controlled release of an effective drug for the treatment of HSV.

2. Description of Related Art

Human herpes simplex virus type-1 (HSV-1) is an alphaherpes virus in the genus Simplexvirus (Strauss, 2002). This is a widely studied virus that is used as a model virus for understanding herpesvirus replication and infection pathways (Strauss, 2002). HSV-1 infects mucosal epithelium and dermal epithelial cells, causing lesions on the epithelium of the face, generally the lips or nose (Strauss, 2002; Whitley, 2001). These blisters are typically termed cold sores or fever blisters. There are, however, other more serious diseases associated with HSV-1, including conjunctivitis, keratitis, hepatitis and encephalitis (Hirsch, 1995; Smith et al., 2001; Tang et al., 1999; Whitley, 2001; Whitley and Roizman, 2001). Additionally, a connection between dementia and HSV-1 is currently being investigated (Dobson et al., 2003; Itzhaki et al., 1997; Jamieson et al., 1991).

While estimates range, it is estimated that 80% of the adult population carries HSV-1, typically asymptomatically (Whitley, 2001; Whitley and Roizman, 2001). Primary infection usually occurs during childhood, and subsequent to the initial outbreak, the virus enters the peripheral nervous system, residing there permanently in a latent state of infection. The typical residence of latent HSV-1 is the trigeminal ganglia, which is the fifth cranial nerve arising from the pons, branching to enervate the face (Baringer and Swoveland, 1973; Shier, 2004; Whitley, 2001). During the latent state, unincorporated viral DNA is present in the nucleus of the majority of the sensory nerve cells of the trigeminal ganglia, but no signs or symptoms of infection are present (Baringer and Swoveland, 1973; Theil et al., 2001).

Many people never see an emergence of the disease from the latent stage of infection; however, others do have recurrent outbreaks. Reactivation occurs in an average of 33% of people infected with HSV-1. Of these people who do see a recurrence, 5% have recrudescence rates of at least one episode per month, 34% have at least one episode every two to eleven months, and 61% have at least one episode per year (Spruance et al., 1977; Whitley, 2001). Reactivation of the virus may be attributed to many factors, including burns, physiological and emotional stress, fever, hormonal changes, and exposure to ultraviolet light (Strauss, 2002; Whitley, 2001). In immune compromised individuals, such as people infected with human immunodeficiency virus (HIV) or organ transplant recipients, outbreaks can occur with increased frequency and are harder to control (Corey and Spear, 1986a; Corey and Spear, 1986b; Erlich et al., 1989).

Several different treatments are available for combating infections with human herpes viruses. Therapies focus on either treatment of acute symptoms or suppression of the virus from reactivation. Most antiherpetic treatments are composed of oral doses of a nucleoside analogue, such as acyclovir (ACV), ganciclovir, valacyclovir or famciclovir. These drugs are very effective, but due to relatively poor bioavailability, require a high level of patient compliance, with many patients taking multiple oral doses daily at set times to obtain relatively constant drug levels. For example, a typical oral dosing of ACV to treat an acute outbreak is 200 mg of the drug five times a day for five days (Tyring et al., 2002).

Compared to treating acute symptoms, complete suppression of any recurrent outbreak is advantageous because it reduces the pain and stress the patient may incur. Preventing lesions from forming also reduces the risk of acquiring another infection, such as HIV, through the broken skin that may occur as the result of recrudescent infection (Hook et al., 1992; Wald and Link, 2002). Towards that goal, ACV has been proved to be capable of not only treating acute HSV-1 infections, but also suppressing reactivation of the virus when taken prophylactically over the long term (Lietman, 1982; Saral et al., 1981).

ACV enters HSV-1-infected cells and is phosphorylated by the viral enzyme thymidine kcinase (Nash et al.) to ACV monophosphate (Furman et al., 1984; Fyfe et al., 1978), which is subsequently modified by cellular kinases to its active form, ACV triphosphate (Miller and Miller, 1982). The HSV-1 DNA polymerase mistakenly incorporates the ACV triphosphate into the growing DNA chain, resulting in premature termination of viral replication due to the lack of a free 3′ hydroxyl group on the drug. ACV is very effective at disrupting viral DNA synthesis without halting host cell DNA synthesis because the viral TK is capable of phosphorylating ACV, whereas the cellular orthologue is not (Furman et al., 1984; Fyfe et al., 1978). Furthermore, the viral DNA polymerase has a higher affinity for ACV-triphosphate (average Ki=3.5 mM), relative to the drug's affinity for host eukaryotic DNA polymerase (Ki=0.96±0.08 uM), providing an even greater effect of the drug in HSV-1-infected cells, while providing an extra margin of safety in normal host cells (Furman et al., 1979; Furman et al., 1984; Martin et al., 1994).

Despite the efficacy of ACV in the treatment of HSV-1, patient compliance and bioavailability are still major issues and, therefore, maintenance of the requisite levels of drug in the patient is a potential problem. It is estimated that the amount of ACV delivered to the bloodstream for humans is only 10-20% of the oral dose taken, with nearly 80% of the consumed ACV removed due to renal clearance (de Miranda and Blum, 1983; Laskin et al., 1982). Because high levels of unutilized drug are excreted unaltered before having a chance to provide antiviral protection (Lasluin et al., 1982; Lietman, 1982), the dose, dosing frequency, and cost of treatment all rise.

Several attempts have been made to increase the bioavailability of ACV, such as the production of valacyclovir, an ACV ester with 1-valine (Tyring et al., 2002; Weller et al., 1993). This prodrug of ACV has increased bioavailability, to about 50% of the oral dose consumed (Tyring et al., 2002; Weller et al., 1993); however, daily dosing is still required. In addition to the esterification of ACV with a valine, Tolle-Sanders and colleagues attempted to increase the oral bioavailability of ACV using ACV linked to chenodeoxycholate via a valine, to form ACV-valylchenodeoxycholate, which is cleaved by a cellular esterase to release ACV (Tolle-Sander et al., 2004). This latter attempt resulted in a two-fold increase in bioavailability (Tolle-Sander et al., 2004). Again the dosage regime is problematic because of the requirement for daily dosage.

Thus, it would be advantageous to provide an effective alternative for delivering an antiviral drug such as ACV that employs the use of a controlled release delivery device. The ideal vehicle would release ACV at a steady and suppressive dose over an extended period of time, thereby obviating the problems with bioavailability, patient compliance, and the requirement for new prescriptions of oral ACV month after month, year after year.

### SUMMARY OF THE INVENTION

The present invention relates to the controlled release of at least one therapeutic compound from an implantable polymeric or polymeric containing substrate, wherein the therapeutic compound is dissolved, distributed, dispersed and/or impregnated into at least the outer surface of the substrate, and more preferably, throughout the substrate.

In one aspect, the present invention relates to an implantable drug delivery device comprising a polymeric or polymeric containing material in combination with a therapeutic compound effective for the treatment of an alpha herpes virus in a mammal including members of the herpesvirus family such as feline or human herpesvirus. Preferably, the therapeutic compound is in an amount that will effectively treat HSV-1, HSV 2 and/or VZV or reduce reactivation, and wherein the implantable drug delivery device is positioned near the site of latent infection. The therapeutic compound may include any effective nucleoside that reduces reactivation of latent infections and/or controls the infection including but not limited to acyclovir (ACV), guanosine, valacyclovir, functional analogues, functional equivalents or a combination thereof.

Preferably, the therapeutic compound diffuses from the polymeric or polymeric containing material into the surrounding biological matrix, thereby providing for a controlled release of the therapeutic compound. The diffusion rate will preferably approach a constant diffusion value equating to a diffusion rate for approaching equilibrium in the surrounding biological matrix at ambient biological temperature, pressure and pH.

In another aspect, the present invention relates to a method for treating or controlling an alpha herpesvirus, the method comprising implanting into a subject, having the need for such treatment, the implantable drug delivery device of the present invention and positioning such device at the local point of infection, or at the site pf observed clinical symptoms, such as the surface epithelium, mucus membrane or cutaneous layer on which the lesions are likely to appear. Notably when the infection is due to HSV, the local delivery is positioned near or at the trigeminal ganglia or other nerves that enervate the facial epithelium.

The drug delivery device is preferably fabricated of a polymeric or a polymeric containing material formed into a substrate that is sufficiently flexible for comfortable inclusion in a subject, yet sufficiently rigid for ease of insertion. Notably, the substrate can be fabricated into any geometric shape that provides sufficient surface area for inclusion and delivery of a therapeutic amount of an active agent found to exhibit antiherpes effectiveness. For example, the geometric shapes for implantation may include rods, bundle of multiple rods, disks, doughnut shapes, helical, elliptical, triangular or oval shapes. Preferably, rods have a diameter from about 1 to 3 mm and can be from about 5 to 20 mm long. Disks can include structure with a thickness of from 1 to 3 mm with a diameter from about 3 mm to 8 mm. Further, the substrate can be fabricated of multiple layers providing different concentrations of the therapeutic compound or in the alternative different therapeutic compounds. The substrate may be fabricated of either biodegradable material or non degradable material. Notably, if the polymeric material is biodegradable then the time span for biodegradability of the material preferably does not exceed the diffusion rate of the therapeutic compound from the polymeric material.

Yet another aspect of the present invention relates to a method of fabricating the drug delivery device of the present invention, comprising the steps of obtaining particles comprising a therapeutic compound for the treatment of HSV and combining at least the therapeutic compound with a polymeric or polymeric containing material in a manner wherein the therapeutic compound is essentially homogeneously dissolved or dispersed in the polymeric material.

In a further aspect, the present invention relates to a method for treating and controlling HSV, the method comprising:
a. implanting subcutaneously a silicone or silicone-based substrate, wherein the substrate comprises a therapeutic compound for the treatment of HSV, wherein the therapeutic compound is ACV and ACV releases from the substrate with near-zero order kinetics regardless of temperature and pH, and wherein the surface area of the substrate is directly proportional to the amount of therapeutic compound released per unit time.

Therapeutic amounts of the therapeutic compound may be in the range from about 0.02 to 50 ug/day and more preferably from about, 1 to 29 ug/day, and most preferably, from about 0.5 to about 2 ug/day. Unexpectedly, the dosage level per day to suppress HSV-1 is significantly lower than currently administered in oral dosage regimes.

Other features and advantages of the invention will be apparent from the following detailed description, drawings and claims.

### BRIEF DESCRIPTION OF THE FIGURES

FIG. 1 A-E show schematic drawings of the implants (not to scale), wherein the light areas indicate silicone mixed with acyclovir; the darker areas indicate drug-free silicone. A. Lateral view and cross section of covered rod; B. Lateral view and cross section of uncovered rod; C. Top and lateral views of covered disk; D. Top view of uncovered disk; E. Photograph of uncovered rod ACV-silicone implant.

FIG. 2 shows a graphical timeline for the amount of ACV released every 24 hours from two covered rod implants, E1 and E2. Quantities of ACV in medium were calculated based on a standard curve.

FIG. 3 shows the average release of ACV from implants every 24 hours for 63 days. Release of ACV (ug) obtained each day from the different styled implants for duration of the study. Insets in each graph show an expanded y-axis for the release kinetics of ACV from days 6 to 63 for each implant type. Analyses were conducted exactly as outlined in the legend to FIG. 2. A. Uncovered rod; B. Covered rod; C. Uncovered disk; D. Uncovered disk; E. A control uncovered rod implant containing no ACV. For part E, raw absorbencies are shown, instead of drug concentrations, as these readings were below the reliable detection threshold of the instrument and below the minimum value for the linear area of the standard curve.

FIG. 4 shows the release of ACV from uncovered rods maintained at 37° C. at three pH levels (A. pH 6.0, B. 7.0, and C. 8.0). Release of ACV (ug) obtained each day from implants for duration of the study are shown for each pH. insets in each graph show an expanded y-axis for the release kinetics of ACV from days 6 to 63 for each implant type. Analyses were conducted exactly as outlined in the legend to FIG. 2. D. Control uncovered rod implants containing no ACV were also made and followed for 37 days at pH 6.0, 7.0, and 8.0. Part D shows the absorbencies at 251 nm. Optical density is shown, instead of drug concentrations, as these readings were below the threshold of the instrument and below the minimum value for the linear area of the standard curve (ACV concentration<0.02 ug/mL).

FIG. 5 shows the release of ACV from uncovered rods maintained at 25° C. at three pH levels (A. pH 6.0, B. 7.0, and C. 8.0). Release of ACV (ug) obtained each day from implants for duration of the study are shown for each pH. Insets in each graph show an expanded y-axis for the release kinetics of ACV from days 6 to 63 for each implant type. Analyses were conducted exactly as outlined in the legend to FIG. 2. Control uncovered rod implants containing no ACV were also made and followed for 37 days at pH 6.0, 7.0, and 8.0. Part D shows the absorbencies at 251 nm. Optical density is shown, instead of drug concentrations, as these readings were below the threshold of the instrument and below the minimum value for the linear area of the standard curve (ACV concentration<0.02 ug/mL).

FIG. 6 shows the release of ACV from uncovered rods maintained at 4° C. at three pH levels (A. pH 6.0, B. 7.0, and C. 8.0). Release of ACV (ug) obtained each day from implants for duration of the study are shown for each pH. Insets in each graph show an expanded y-axis for the release kinetics of ACV from days 6 to 63 for each implant type. Analyses were conducted exactly as outlined in the legend to FIG. 2D. Control uncovered rod implants containing no ACV were also made and followed for 37 days at pH 6.0, 7.0, and 8.0. Part D shows the absorbencies at 251 nm. Optical density is shown, instead of drug concentrations, as these readings were below the threshold of the instrument and below the minimum value for the linear area of the standard curve (ACV concentration<0.02 ug/mL).

FIG. 7 illustrates photographs showing Vero cells 48 hours after either mock infection or HSV-1 KOS infection. A-C. mock infected Vero cells; D-F. HSV-1 infected Vero cells. Cells were treated with either no active agent (A and D), 0.22 ug ACV (B and E), or two silicone implants (C and F). Note arrows in part D pointing to regions of the culture showing CPE that is characteristic of HSV-1 infection.

FIG. 8 shows the level of ¹⁴C-labeled acyclovir (0.625 μCi/12 mg/ACV/Implant) in proximal skin samples.

FIG. 9 shows the level of ¹⁴C-labeled acyclovir (0.625 μCi/12 mg/ACV/Implant) in distal skin samples.

FIG. 10 shows the level of ¹⁴C-labeled acyclovir (0.625 μCi/12 mg/ACV/Implant) in brain tissue.

FIG. 11 shows the level of ¹⁴C-labeled acyclovir (0.625 μCi/12 mg/ACV/Implant) in spinal cord.

FIG. 12 shows the release of guanosine from a 2 mm implant.

FIG. 13 shows the results of decreasing diameter of the implant with the concomitant increase of guanosine release.

FIG. 14 shows that a 1 mm implant comprising Valacyclovir has a variable release pattern and limited structural integrity.

FIG. 15 shows that a 2 mm implant comprising Valacyclovir has a variable release pattern and limited structural integrity.

FIG. 16 shows that a 1 mm implant comprising ACV exhibits similar release pattern as a 2 mm diameter ACV implant.

FIG. 17 shows comparative percentages of reactivation of latent HSV-1 infections.

### DETAILED DESCRIPTION OF THE INVENTION

Definitions

The terms “subject,” “individual,” and “patient,” as used herein, are used interchangeably and refer to any subject, generally a mammal (e.g., human, canine, feline, equine, bovine, rodent, etc.), in which drug delivery is desired.

The term “therapeutically effective amount,” as used herein, is meant an amount of a therapeutic agent, or a rate of delivery of a therapeutic agent, effective to facilitate a desired therapeutic effect.

The term “functional equivalent” means that the compound retains some or all of the biological activity of the corresponding compound.

The term “functional analog,” as used herein means compounds derived from a particular parent compound by straightforward substitutions that do not result in a substantial (i.e. more than 100×) loss in the biological activity of the parent compound, where such substitutions are modifications well-known to those skilled in the art.

The terms “treatment” and “treating,” as used herein, refer to obtaining a desired pharmacologic and/or physiologic effect. The effect may be prophylactic in terms of complete or partial prevention of a disease or symptom thereof and/or may be therapeutic in terms of a partial or complete cure for a disease and/or adverse affect attributable to the disease.

The term “drug delivery device,” as used herein, is meant to encompass any device that can retain a quantity of drug and that facilitates movement of drug from the drug delivery device to a site external to the drug delivery device. “Drug delivery device” thus encompasses controlled drug release devices, as well as devices that release drug in an unpatterned (e.g., substantially unregulated) manner.

The term “controlled release,” as used herein, is meant to encompass release of substance (e.g., a drug) at a selected or otherwise controllable rate, interval, and/or amount. “Controlled release” thus encompasses, but is not necessarily limited to, substantially continuous delivery, patterned delivery (e.g., intermittent delivery over a period of time that is interrupted by regular or irregular time intervals), and delivery of a bolus of a selected substance (e.g., as a pre-determined, discrete amount of a substance over a relatively short period of time (e.g., a few seconds or minutes).

The term “layered,” as used herein, is meant to encompass a substrate that may include different compounds, concentrations of compounds, or polymeric or polymeric containing materials.

The term “biodegradable,” as used herein, refers to a material that is dissolvable in physiological conditions by physiological enzymes and/or chemical conditions.

The terms “drug formulation,” “formulation,” and “drug,” as used herein, are used interchangeably and meant to encompass any substance suitable for delivery to a subject, which substances can include pharmaceutically active drugs, as well as biocompatible substances that do not exhibit a pharmaceutical activity in and of themselves, but that provide for a desired effect at a treatment site, e.g., to flush or irrigate a treatment site (e.g., saline).

The present invention provides a drug delivery device adapted to release, primarily by equilibration, relatively low dose levels of a therapeutic compound in a controlled release fashion. The present drug delivery devices are designed to strike a balance between the high volumetric loading required for drug release by diffusion from a porous matrix, and the low volumetric loading required for drug release at low dose levels. This is achieved by loading the polymeric or polymeric containing material with at least one therapeutic compound. The release profile preferably, has zero order release kinetics similar to those for osmotic excipient release profiles.

Notably, however, because the therapeutic compound is present at only low levels within the polymeric material, drug release is limited, at a predominantly constant rate, to low dose levels.

In one embodiment, polymers appropriate for use as matrix in the present delivery devices are biocompatible and generally resistant to chemical attack by the environment of use so that the matrix remains intact during the period of drug release. Suitable for use as polymeric matrix in the present delivery devices are the elastomers, such as ethylene-vinyl ester copolymers, including ethylene-vinyl acetate copolymer. Acceptable properties are also exhibited by silicone, and silicone-based elastomers, such as polydimethylsiloxane.

Silicone is an elastomeric polymer that is non-biodegradable. There are both advantages and disadvantages for having such an implant that does not degrade in the human body. First, there are no unwanted side effects, such as drug interactions or toxicity to the host, associated with ACV. Furthermore, silicone is considered safe and generally biocompatible and, most importantly, already approved by the US FDA for use in medical devices, including controlled release delivery vehicles, although a rare adverse patient reaction may occur against the silicone (Park and Park, 1996; Tiffany, 1995). The most straightforward shortcoming is that non-biodegradable implants must be removed. Removal of a silicone-ACV implant is optional, but not necessary.

Silicone is an excellent polymer for delivering therapeutics, and has a long history of safety and efficacy in the clinic. For example, silicone has been used in the delivery of levonorgestrel in a product marketed as Norplant, for the long-term prevention of pregnancy (Robertson et al., 1983; Sivin et al., 1997). Compudose® is another implant which uses silicone to deliver estrogen to cattle for improving growth (Ferguson, 1988). Additionally, silicone has been used as a drug-releasing polymer in cardiovascular, bronchial, and ureteral stents (Chew et al., 2004; Chew, 2004; Khanavkar and Ewig, 2004). Maeda et al. developed a silicone-based delivery device for the antiviral compound ganciclovir; this research was aimed at delivering constant levels of the drug for suicide gene therapy for brain tumors (Maeda et al., 2002).

To prepare the delivery device, therapeutic compounds, preferably in particle form, are dispersed within the selected polymeric material using a protocol that is dictated primarily by the choice of polymer. Once the therapeutic particles are prepared and size-selected, they are dispersed uniformly within the selected polymer to achieve a volumetric loading which is appropriate for a porous network to form to provide for movement of the particles through the substrate and appropriate for the dosage level desired.

To achieve appropriate volumetric loading, particles and polymeric material in either cured or uncured state are mixed simply in the desired volumetric ratio. To prepare matrices based on silicone, for example, dispersion is achieved while the polymer is in the uncured state. In particular, particles are mixed with the polymer components by repeatedly spreading a film of the mix onto glass or other suitable surface, with care to avoid air entrapment. The mixture having uniformly dispersed particles can then be pressed into sheets e.g. 0.5 mm to about 2.5 mm thick for stamping into a disk or added to a plastic mold to form rods, and then allowed to cure.

In a preferred embodiment of the invention, the particles may be dispersed uniformly within a polymeric material, such as silicone. Particle dispersion can be effected either by solvent casting, or by the precipitation method exemplified herein. The solvent casting approach entails mixing appropriate volumes of particles and polymeric material in a solvent, such as methylene dichloride, and then casting the vigorously agitated suspension in a cold mold. The impregnated polymer solidifies on contact, and is then gradually warmed to room temperature over a period of about two days. A degassing step is desirable; to ensure complete removal of trapped air bubbles. Then the polymeric material may be shaped as appropriate for administration to the patient by the chosen route of administration.

Shaping can be achieved by any conventional means, such as by extrusion, injection molding or by melt press. For example, the shape of the device may include cylindrical, bullet, elliptical, circular, bulbuous, loop or any other shape suited for placement in the biological environment.

In some embodiments, the particles or therapeutic compounds dissolved or dispersed in the polymeric material may include one or more additional components that function to limit the rate of diffusion of the therapeutic compound from the substrate to the surrounding biological matrix. Non-limiting examples of matrices are gels of biopolymers i.e. alginates, gelatins, chitin, polyvinylpyrrolidone (PVP), collagen, polylacticpolyglycolic acid, and polyanhydride. Other non-limiting examples of controlled-release matrices are polyurethane, dimethylpolysiloxane, ethylene vinyl acetate, polymethyl methacrylate, polyamide, polycarbonate, polyester, polyethylene, polypropylene, polyhydroxybutyrate, polystyrene, polyvinyl chloride, polytetrafluoroethylene, and cellulose acetate.

In other embodiments, the formulation may include one or more additional components that function to increase the rate of diffusion of the therapeutic compound from the substrate to the surrounding biological matrix. Inclusion of a water-swellable polymer in the substrate may increase the rate of release of the therapeutic compound per unit time, and in thus the total quantity of therapeutic compound released. Examples of water-swellable polymers which may be included are products such as galactomannans, cellulose products, tragacanth, polyglycosides, polyvinylpyrrolidones, finely powdered polyamides, water-soluble polyacrylamide, carboxyvinyl or polymerisates.

The present invention provides methods of treating a subject with an alphaherpes virus by administering a therapeutic compound via drug delivery device according to the invention. In operation, a substrate of the invention is implanted into a subject at or near the local point of infection, or at the site of observed clinical symptoms, such as the surface epithelium, mucus membrane or cutaneous layer on which the lesions are likely to appear. Preferably, if the virus is HSV-1, the delivery device is placed at or near the trigeminal ganglia, sacral, dorsal root ganglia or other nerves that enervate the facial epithelium.

The delivery device of the invention can be used alone or as an adjunct to other therapeutic regimen (e.g. oral or intravenous therapy, etc.).

Release characteristics of at least one therapeutic compound from the substrate into a surrounding medium may depend upon the level of loading. Thus, the loading levels can be below the saturation level of the therapeutic compound in the substrate, at the saturation level or above the at saturation level. When loading is below the saturation limit of the therapeutic compound, release is dominated by diffusion of the therapeutic compound through the polymeric or polymeric material. Release of hydrophobic active compounds into biological systems, which is typically of an aqueous nature, begins slowly. Thereafter, release can remain very slow.

Alternatively, when loading of the therapeutic compound is above or greater than the saturation limit, release from the substrate occurs first at the substrate surface where surface bound therapeutic compound is simply dissolved and released, leaving a cavity or porous structure. This cavity or porous structure, in turn, exposes deeper layers of the therapeutic compound to the ingressing fluids of the surrounding medium resulting in dissolution and release of the therapeutic compound. As this process continues over time, the polymeric or polymeric containing substrate assumes a more porous structure. Release of subsequently exposed internal layers of the therapeutic compound within the substrate requires that the therapeutic compound diffuses through a porous and tortuous path to the exterior. The release of hydrophilic therapeutic compounds into the surrounding biological matrix typically begins very quickly and slows as that release from the interior of the substrate due to the tortuous path to the exterior of the substrate.

The present invention relates to a drug delivery device that provides for a controlled release of a therapeutic compound. In a preferred embodiment, the drug delivery device is fabricated from a silicone polymer or a silicone containing polymeric material in combination with at least one therapeutic drug compound, wherein the therapeutic compound is effective against HSV and other alpha-herpes viruses and is suspended, dissolved, dispersed or impregnated within the polymer and the device provides for the controlled release of such a therapeutic compound into a surrounding physiological solvent.

ACV is a very effective, safe, and inexpensive drug capable of controlling alphaherpes infections and the present invention provides for a long-lived subcutaneous device that releases a controlled levels of ACV.

The ACV implant developed here is aimed at suppressing reactivation of an alpha virus including herpesviruses infecting both humans, such as HSV-1, and animals, such as feline herpes virus. However, it should be noted that other herpesviruses, including herpes simplex virus type-2 (HSV-2), the etiologic agent of genital herpes, and Varicella-Zoster virus (VZV), which causes chickenpox and shingles, are both susceptible to ACV therapy (Strauss, 2002; Whitley, 2001). Approximately 89% of people infected with HSV-2 had reactivation events after a symptomatic primary infection (Benedetti et al., 1994). Of these people, 38% had six and 20% had ten or more reactivation events in the first year after primary infection (Benedetti et al., 1994), a condition for which daily oral acyclovir or valacyclovir therapies have been proved effective. These therapies include once or twice daily doses of the drug and are effective at preventing reactivation for one year or longer (Reitano et al., 1998).

Long-term suppression of these other herpesviruses will not be trivial. The systemic therapeutic quantities of ACV for these viruses, 0.36-0.84 ug per mL in the plasma, are higher than the amounts required to suppress HSV-1 (Crumpacker et al., 1979). HSV-2 is latent in the sacral ganglia, while VZV is primarily latent in the dorsal root ganglia (Whitley, 2001). Therefore, implantation of several ACV implants near the pelvis or the spine may allow for suppressive therapy of these human herpesviruses as well. However, it should be noted that our primary goal is still geared towards treating HSV-1 infections, and managing these other infectious agents using the same ACV-silicone delivery scheme may not be as straightforward.

The use of suppressive therapy could potentially allow for a decrease in the overall prevalence of HSV-1, and potentially HSV-2, in the population, although this is yet to be demonstrated conclusively. It has been shown that the use of acyclovir as a daily suppressive therapeutic agent reduces HSV-2 shedding (Gupta et al., 2004; Wald et al., 1996). Additionally, it has been demonstrated that the use of long-term suppressive valacyclovir therapy reduces the amount of viral shedding and decreases the transmission of HSV-2 to susceptible sexual partners (Corey et al., 2004; Gupta et al., 2004; Miller et al., 2005). Therefore, the use of the ACV-silicone implant may lead to a decrease in transmission rates of both HSV-1 and HSV-2, reducing both the incidence and prevalence of these common pathogens.

The development of an ACV-polymeric implant capable of suppressing alpha herpesvirus reactivation would improve the quality of life for many patients. Additionally, this implant may also help prevent the spread of alpha herpesvirus infections, and allow for control of these infections in patients who are unwilling or incapable of compliance. A controlled release device for antiviral intervention that is popular and beneficial to an extremely large group of people may also lead to a change in the way other pharmaceuticals are delivered to patients.

### EXAMPLES

Exemplary, non-limiting examples and embodiments of the invention will now be described with reference to the figures.

The implantable drug delivery devices of the present invention were evaluated for release kinetics of ACV in response to i) varying amounts of exposed surface area, ii) differing temperature, iii) differing pH levels of the release media into which the implants were placed, and iv) and in vivo testing. Additionally, it was found that these ACV-silicone devices are capable of protecting Vero cells from HSV-1-induced cytopathic effect (CPE) in culture.

Materials and Methods

Implant Development

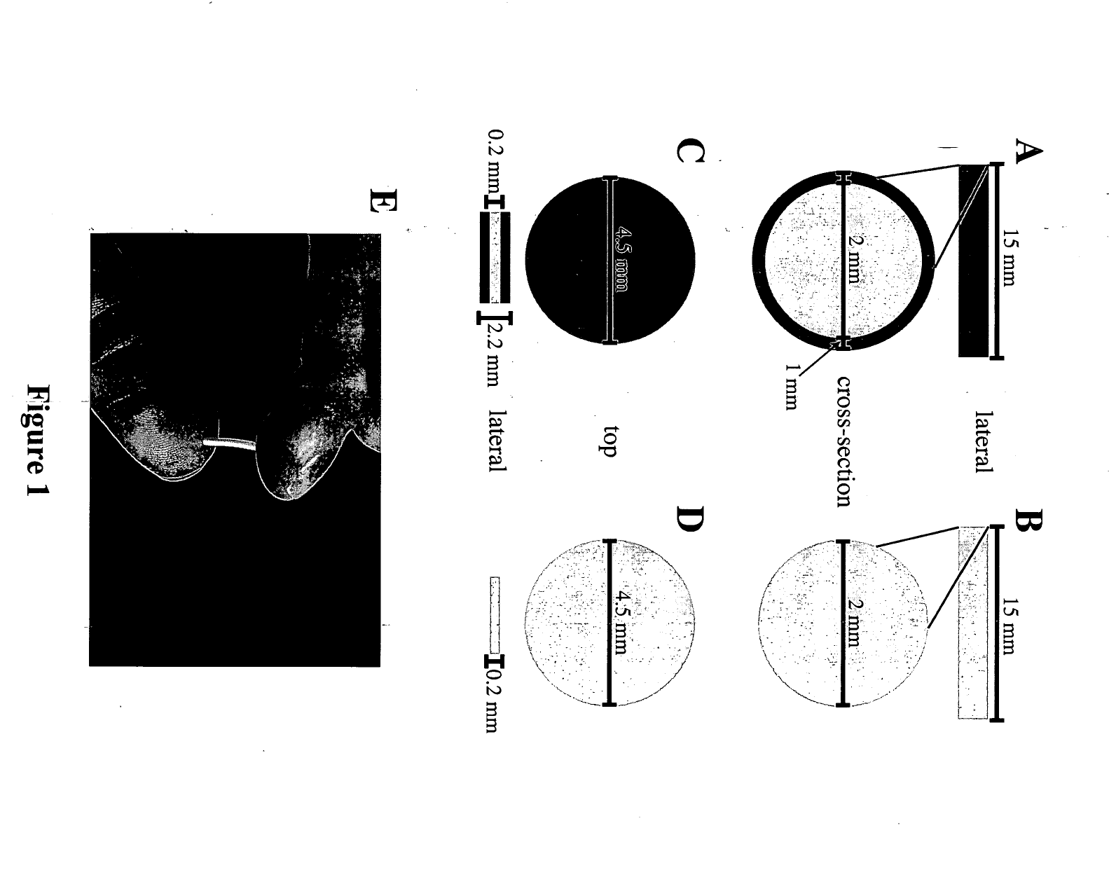

Implants were composed of a matrix of silicone and powdered acyclovir (ACV) (Sigma-Aldrich, St. Louis, Mo.), combined and molded into four different structures, some of which were covered with drug-free silicone. The four implant types developed were a silicone-covered rod, an uncovered rod, a silicone-covered disk, and an uncovered disk (FIG. 1).

The silicone elastomer used was Nusil Med-4050 (Nusil Silicone Technology, Carpinteria, Calif.). Med-4050 is a two-part kit, parts A and B, which are composed of dimethyl and methylvinyl siloxane copolymers with silica. It is a restricted polymer for implantation up to 29 days (Nusil, MED-4050 Product Profile, 2004). However, MED-4050 is similar in all properties to MED-4750, an unrestricted polymer approved for long-term implantation. Parts A and B of MED-4050 are typically mixed in a 1:1 ratio for the final polymer (Nusil, MED-4050 Product Profile, 2004). Part A contains a platinum catalyst and part B contains cross-linkers with silicone hydride groups. When the two parts are mixed, the silicone vinyl and silicone hydride groups become cross-linked through a platinum catalyst-induced reaction (Kajihara et al., 2001; Kajihara et al., 2003; Kajihara et al., 2000; Maeda et al., 2003; Maeda et al., 2004; Maeda et al., 2002).

ACV implants were produced by mixing equal quantities of parts A and B of Med-4050 (0.20 g each) and 0.20 g of ACV. After Parts A and B were each softened individually with ten passes on a chilled two-roll mill press (Keilen Ltd., Terre Haute, Ind.), they were milled together for twenty passes on the press to begin the polymerization process. ACV was then added to the mixture and milled for twenty more passes. Following homogenization, the material was then formed into implants.

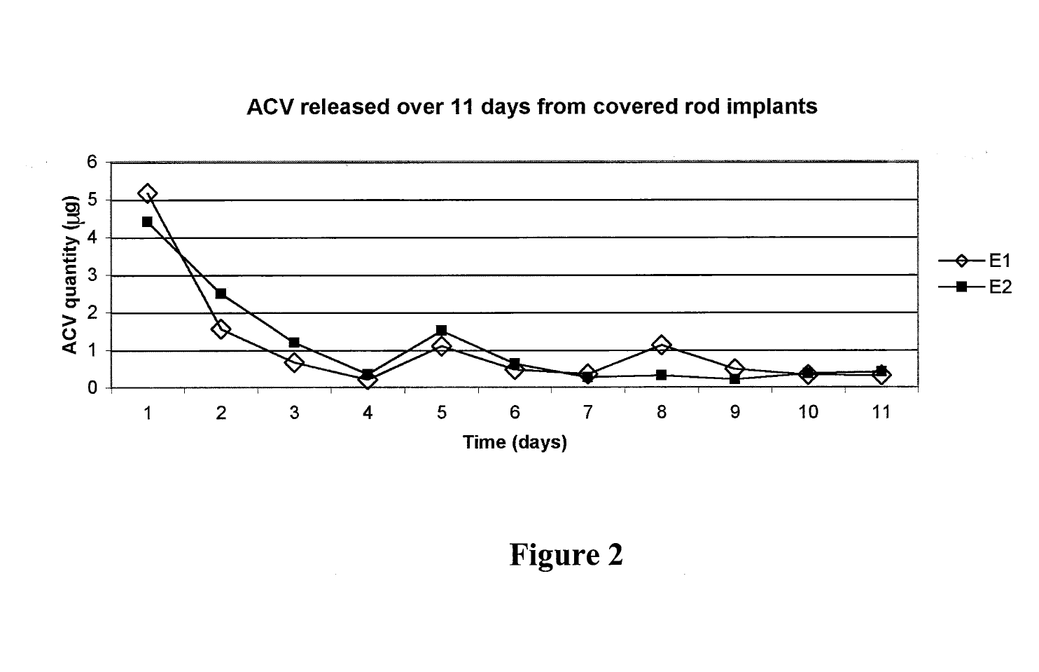

To form rod-shaped implants, the ACV-silicone mixture was loaded into a glass syringe with an internal diameter of 2 mm and extruded through the syringe to form a rod with the same diameter (FIG. 1). A separate mixture (10 g each part Med 4750) was also prepared and pressed into a 1-mm thick sheet for use as the coat of the covered rod implants. These outer sheets were then wrapped around the lateral surfaces of the rod by hand without overlap. Implants were cured at room temperature for 7 days and then at 60° C. for 24 h. After the curing process, implants were cut to 15 mm in length (FIGS. 1 and 2). Disk-shaped implants (FIG. 1) were produced by pressing the drug and silicone mixture into a 0.2-mm thick sheet. The drug-free silicone was pressed into a 1-mm thick sheet. Disks were cut from the silicone sheets using a 4.5-mm diameter stainless steel corer. To form the covered disk implant, the matrix disk was covered on both surfaces with drug-free silicone sheets. Disk-shaped implants, covered and uncovered, were cured as above.

The exposed surface areas of each matrix that contained ACV were 2.86 mm² for the covered disk, 6.28 mm² for the covered rod, 34.62 mm² for the uncovered disk, and 100.48 mm² for the uncovered rod (FIG. 1).

Determining In Vitro Release Kinetics

The rate of release of ACV from the implants was determined in vitro using a spectrophotometer. Implants were placed into 1.5-mL microcentrifuge tubes containing 1.0 mL of phosphate buffers with 0.9% NaCl (phosphate-buffered saline; PBS) (HyQ PBS, HyClone, Logan, Utah) at pH 7.5 and held at 25° C. Every 24 h for 60 days each implant was moved into a new microcentrifuge tube containing 1 mL PBS. Each sample of PBS was then concentrated ten-fold by drying completely in a speed vacuum (Thermo Savant, Waltham, Mass.); dried solute was then vigorously re-suspended with 100 uL distilled water. Spectrophotometry was performed at 251 nm, the λmax of ACV, and compared to standard curves of ACV in each concentrated PBS solution to determine the quantity of ACV in each sample. These data were graphed over time to determine the release kinetics of ACV from each implant under each condition.

To determine if the rate of release of ACV from the silicone was affected by temperature or pH, uncovered rod implants were utilized. Uncovered rod implants were maintained in PBS buffered to three different pH levels (6.0, 7.0 and 8.0) and kept at either 4° C., 25° C. or 37° C. for 37 days. A total sample size of 27 implants was used, which allowed for three replicates per cell (each pH and temperature level combination). ACV release kinetics were determined as above.

Determining Efficacy of Implants at Preventing Cytopathic Effect in Cell Culture

A six-well culture plate was prepared with 4 mL of Dulbecco's modified Eagles' medium (DMEM) (Invitrogen) supplemented with 10% fetal bovine serum and 1% penicillin-streptomycin-Amphotericin B solution (Mediatech) per well. Two rod-shaped implants were added to each of two wells; 0.22 ug of ACV was added to each of two other wells. The plate was incubated for two days at 37° C. and 5% CO2. Vero cells (ATCC CCL-81) were then added to each well and allowed to grow for another 48 hours until almost confluent. HSV-1 KOS (9.0×10³ pfu) (ATCC VR-1493) was added to one of each of the paired wells, and 24 and 48 hours later the cells were photographed with a Nikon D1 2.6 megapixel digital camera, set in black and white mode, attached to an Olympus CK-40 microscope. The resulting digital images were compiled for presentation with Adobe Photoshop.

Release Kinetics from Covered Rods

Two covered rod-shaped implants (E1 and E2), 1 mm diameter and 15 mm long, were initially tested in PBS for 11 days. Implants were suspended in 1 mL of PBS in a 1.7 mL microcentrifuge tube. Every 24 hours implants were placed in fresh release medium. Spectrophotometric analysis of the release medium was performed at 251 nm after the samples were concentrated ten-fold. These implants released ACV continuously for 11 days (FIG. 2). The total amount of ACV released from the implants was 11.75 ug for E1 and 12.14 ug for E2 over the time of the experiment. The implants released varying amounts of drug each day, ranging from 5.17 ug on day one (1) to 0.21 ug on day four for E1, and from 4.41 ug on day one to 0.21 ug on day nine for E2. These days represent the upper and lower limits in the range of drug released during the 11 day trial. These data showed that ACV could be releases from these implants and that the amount of ACV released could be measured using spectrophotometry.

Effect of Exposed Surface Area and Shape on Release Kinetics

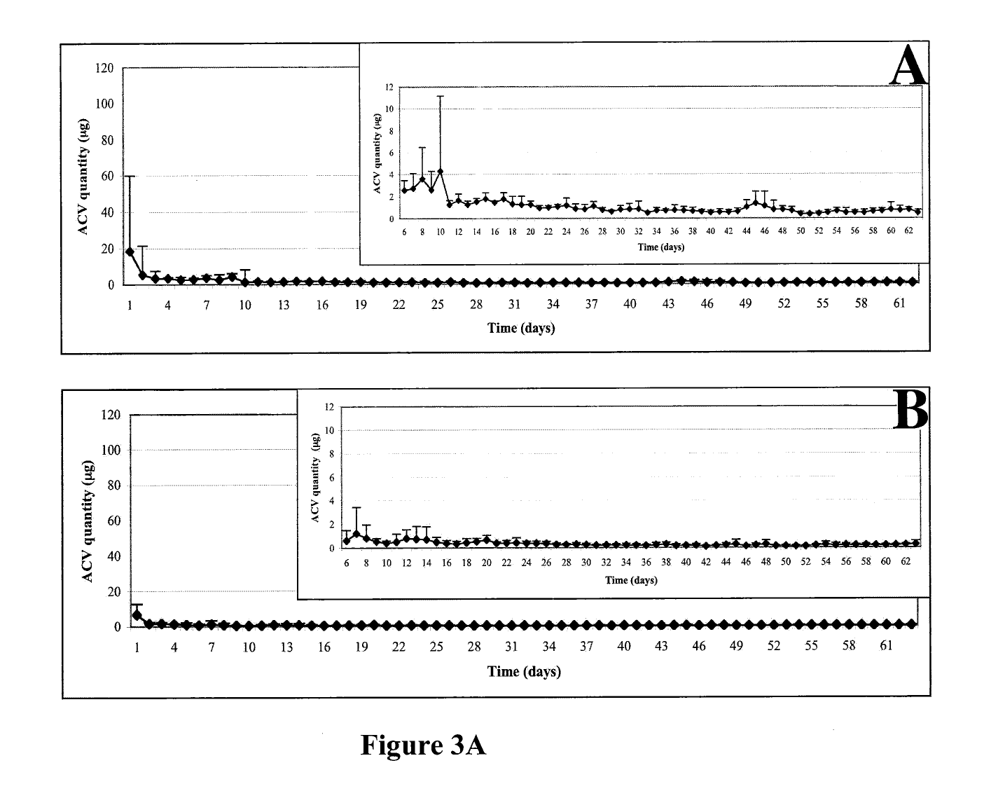

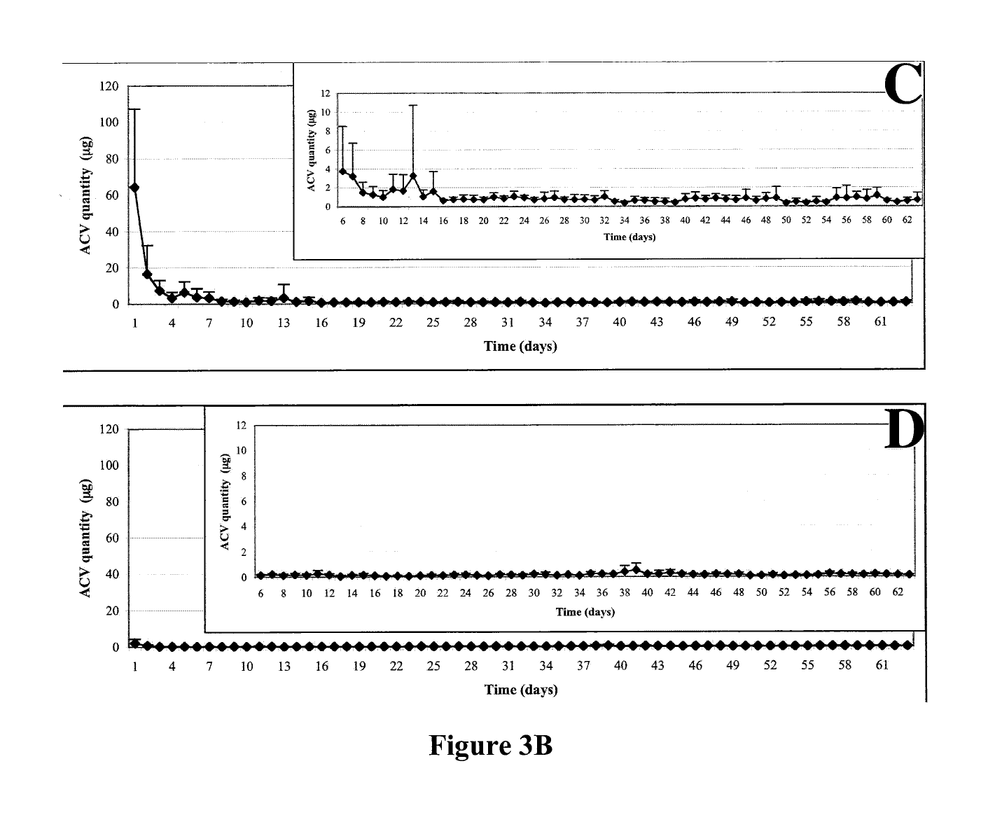

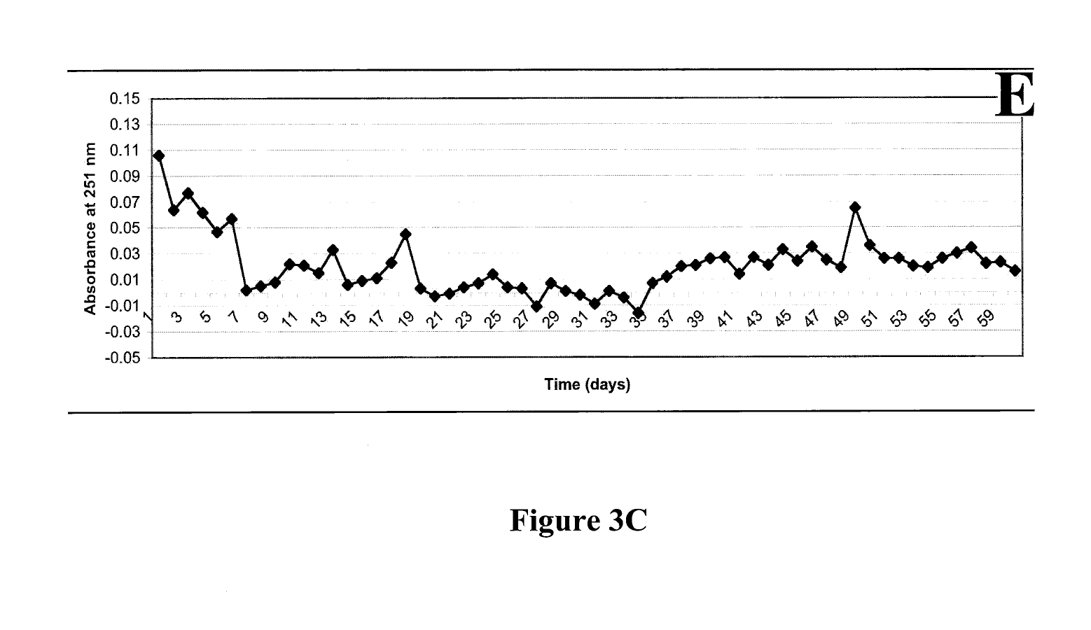

ACV was continuously released from different-shaped implants over a second, 63-day trial period. A burst period of ACV lasting 5 days, which was marked by high variability within the replicates, was seen from the implants (FIG. 3). After the burst period, the release levels of ACV were more consistent between replicates. Therefore, statistical analyses were performed excluding the highly variable burst period data.

The exposed surface area of the matrix, which differs based on the shape of the implant produced did have a significant impact on the release kinetics of ACV from the four different implants (One Way ANOVA with Repeated Measures, df=3.0, P<0.0001). Analysis of the exposed surface areas indicated that the two types of covered implants had considerably lower drug release when compared to the two types of uncovered implants after the initial 5 day burst period (FIG. 3).

The uncovered rods released a total of 207.61±15.10 ug over the 63 days, with an average of 1.07±0.76 ug per day after the initial 5 day burst period (FIG. 3A), whereas the covered rod released a total of 31.52±1.11 ug over the trial, with an average of 0.33±0.21 ug per day after the first five days (FIG. 3B). The uncovered disk released a total of 157.00±9.47 ug over the trial, with an average of 0.93±0.66 ug per day after the first 5 days (FIG. 3C), whereas the covered disk released a total of 13.13±0.36 ug over the time of the trial, with an average of 0.17±0.08 ug per day after the initial burst (FIG. 3D). The two uncovered implants, the uncovered rod and the uncovered disk, did not have significantly different release kinetics (compare 1.07±0.76 ug per day v. 0.93±0.66 ug per day; One Way ANOVA with Repeated Measures, df=1.0, P=0.148); a similar relationship was observed with the two covered implants (compare 0.33±0.21 ug per day v. 0.17±0.08 ug per day). The uncovered rod was selected for further analysis. As a control, implants formed with silicone only in an uncovered rod displayed no measurable ACV release when followed for 60 days (FIG. 3E).

Effect of Temperature and pH

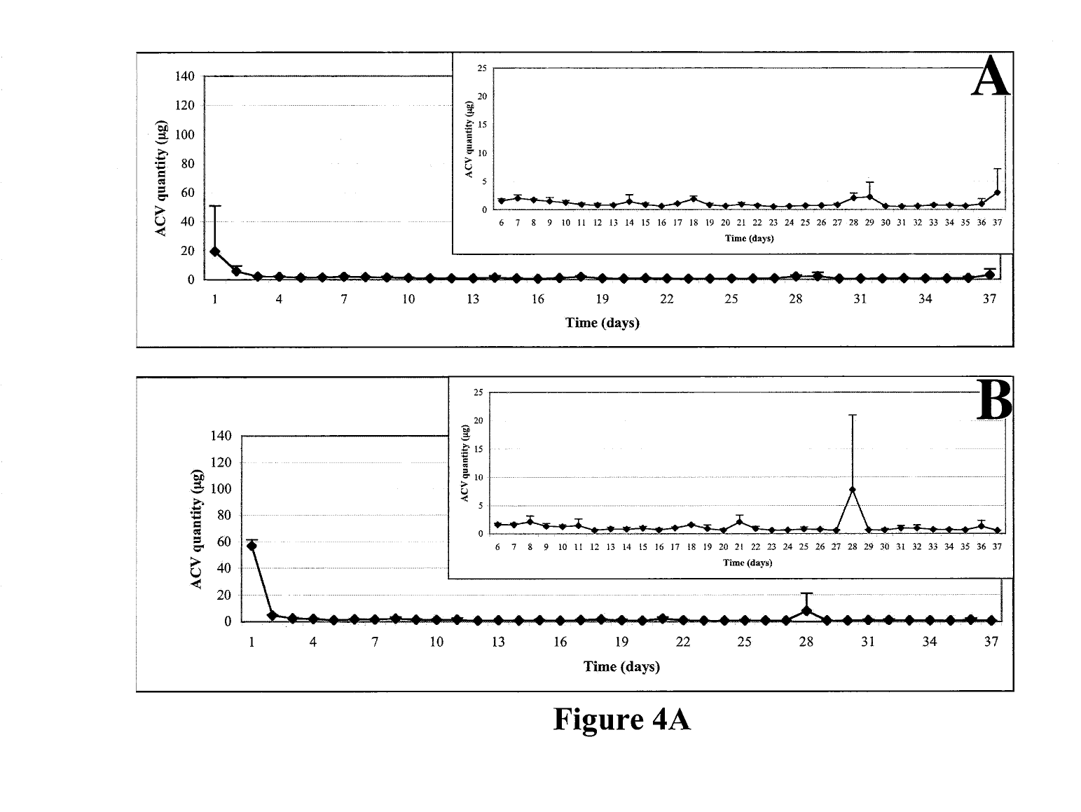

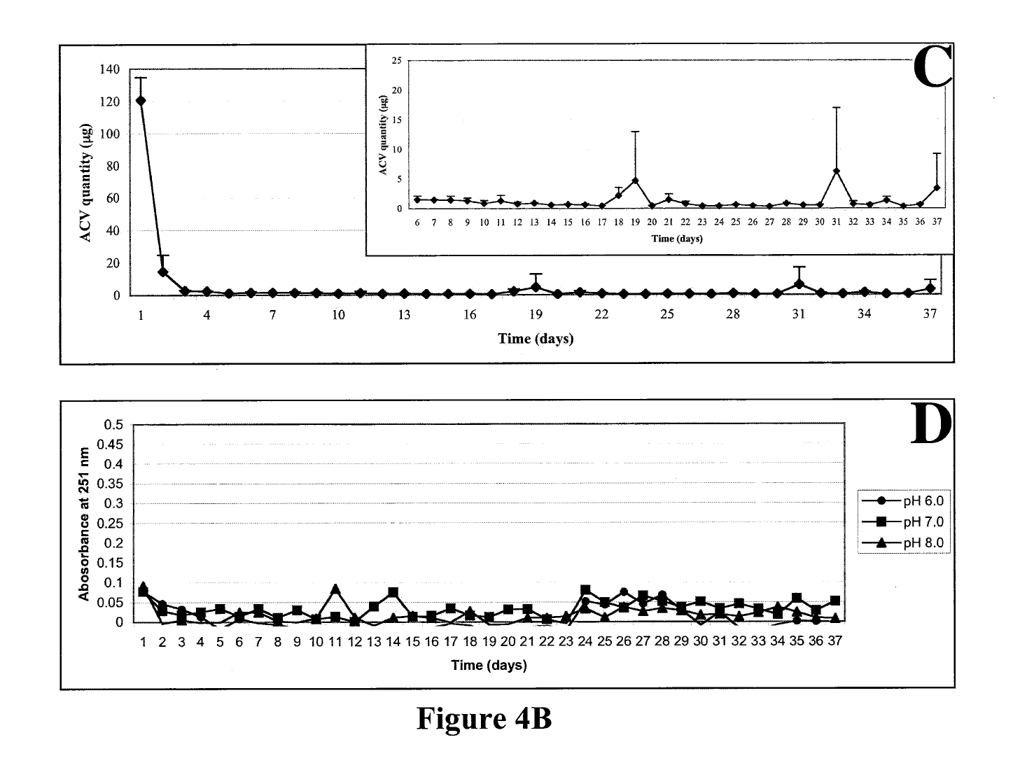

Consistent with previous observations, a burst period of 5 days, marked by high variability within the replicates, was seen in the uncovered implants used for this arm of the study (FIGS. 4, 5, and 6), testing the effects of temperature and pH on the release kinetics of ACV. Because release levels became more consistent within groups after this 5-day burst, statistical analyses were performed after the highly variable burst period, as was conducted previously. The data from this 5-day period, although shown in each figure, is not considered in the descriptions of average daily release in this section.

Release kinetics of implants held at 37° C. showed virtually no difference due to pH treatment (FIG. 4). The uncovered rods at pH 6.0 released a total of 66.10±4.86 ug over the 37-day trial, with an average of 1.10±0.61 ug released per day after the initial 5 day burst period (FIG. 4A), while the implants at pH 7.0 released a total of 104.85±9.30 ug over the trial, averaging of 1.19±1.27 ug released per day (FIG. 4B), and the implants at pH 8.0 released a total of 180.34±19.74 ug over the trial, with an average of 1.22±1.3 ug released per day (FIG. 4C).

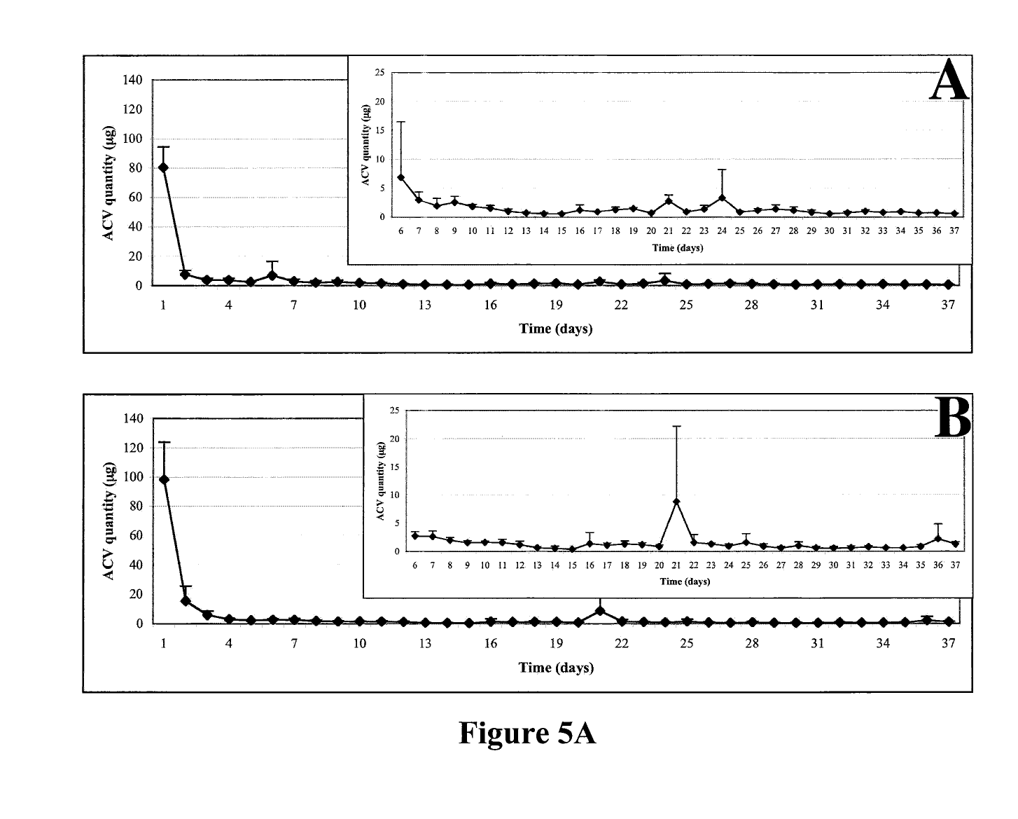

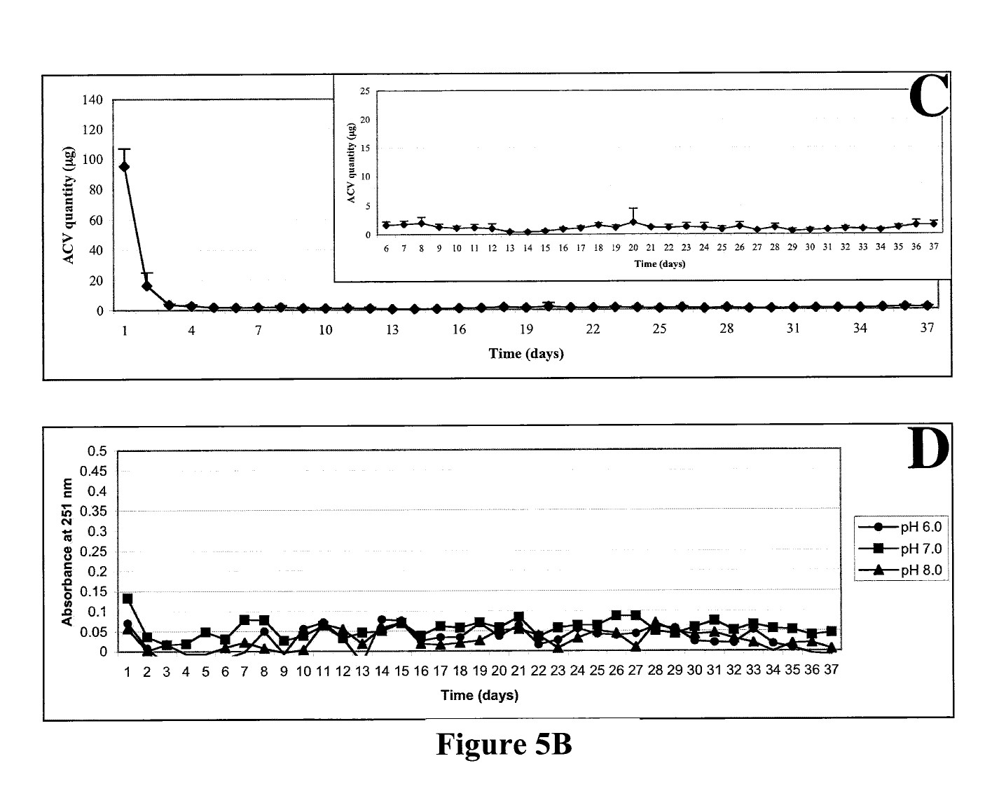

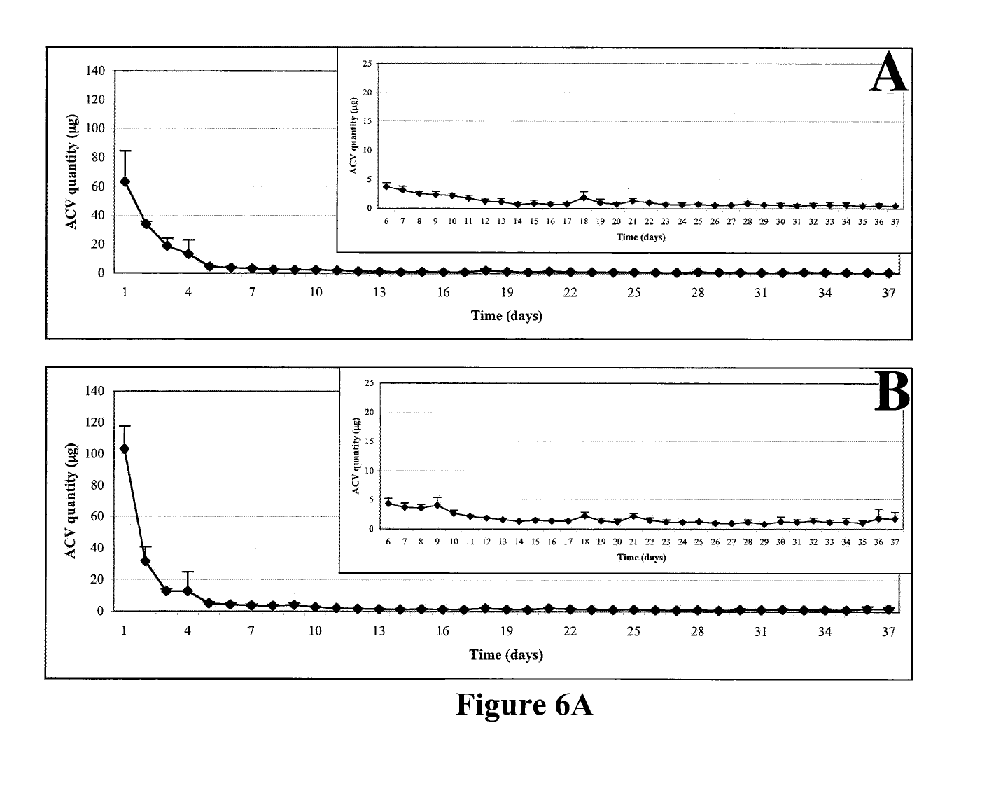

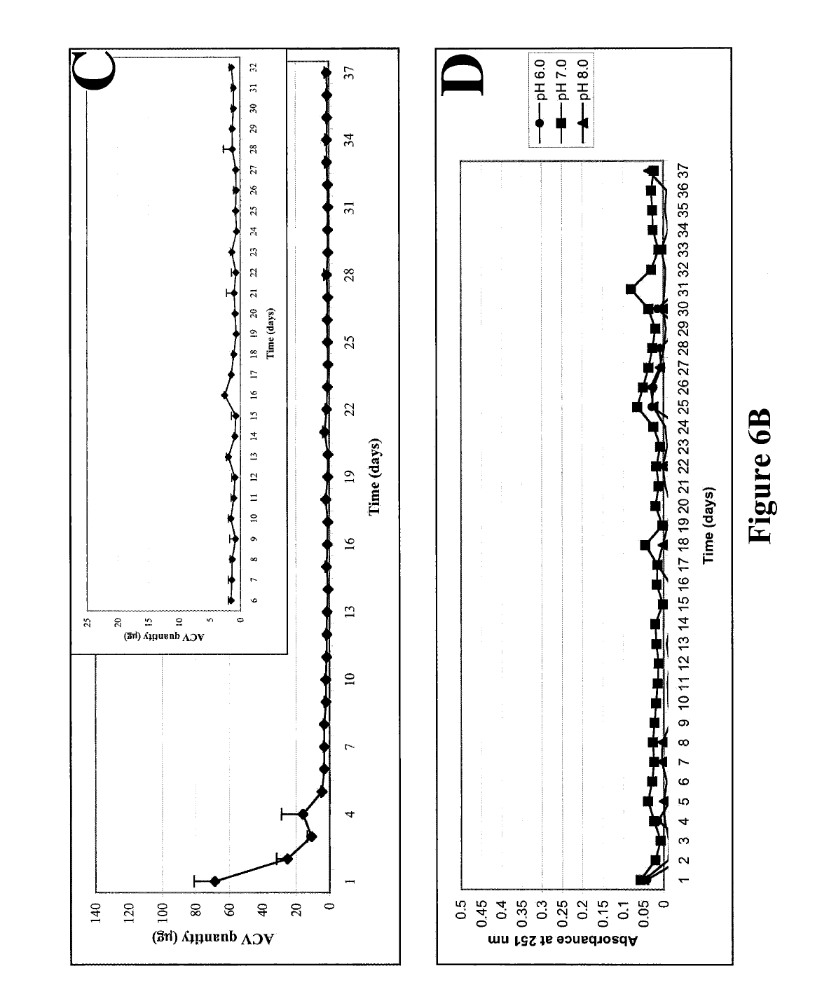

Implants maintained at 25° C. exhibited a similar pattern. The uncovered rods at pH 6.0 released a total of 143.67±13.09 ug, with an average of 1.43±1.23 ug released per day (FIG. 5A); the implants at pH 7.0 released a total of 170.69±16.34 ug over the trial, with an average of 1.42±1.47 ug released per day (FIG. 5B); and the implants at pH 8.0 released a total of 156.57±15.54 ug over the trial, averaging of 1.14±0.42 ug released per day (FIG. 5C). Finally, the uncovered rods maintained at 4° C. and at pH 6.0 maintained released a total of 170.35±12.01 ug over the trial, with an average of 1.13±0.83 ug released per day (FIG. 6A); the implants at pH 7.0 released a total of 220.65±17.33 ug over the trial, with an average of 1.73±0.92 ug released per day (FIG. 6B); and the implants at pH 8.0 released a total of 169.71±11.95 ug over the trial, with an average of 1.39±0.74 ug released per day (FIG. 6C).

There was no difference found in the mean quantity of ACV released per day with respect to temperature (Two Way ANOVA with Repeated Measures, df=2.0, P=0.139) or to pH (Two Way ANOVA with Repeated Measures, df=2.0, P=0.148). Additionally, no significant interaction between pH and temperature was found (Two Way ANOVA with Repeated Measures, df=2.0, P=0.185). Nine controls, made from drug-free silicone, one for each pH-temperature variation, were used with implants of the same dimensions (FIGS. 4D, 5D, and 6D, respectively). Based on absorbance at 251 nm, there was no observable release of ACV from these empty implants.

Effect of Implants on Cytopathic Effect in Cell Culture.

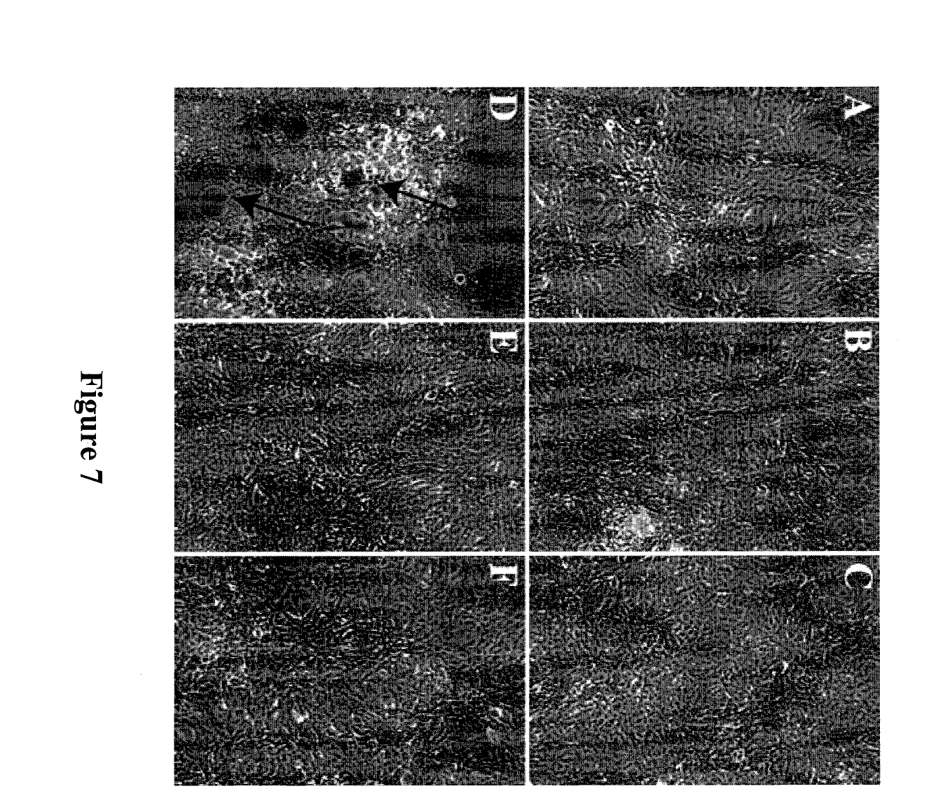

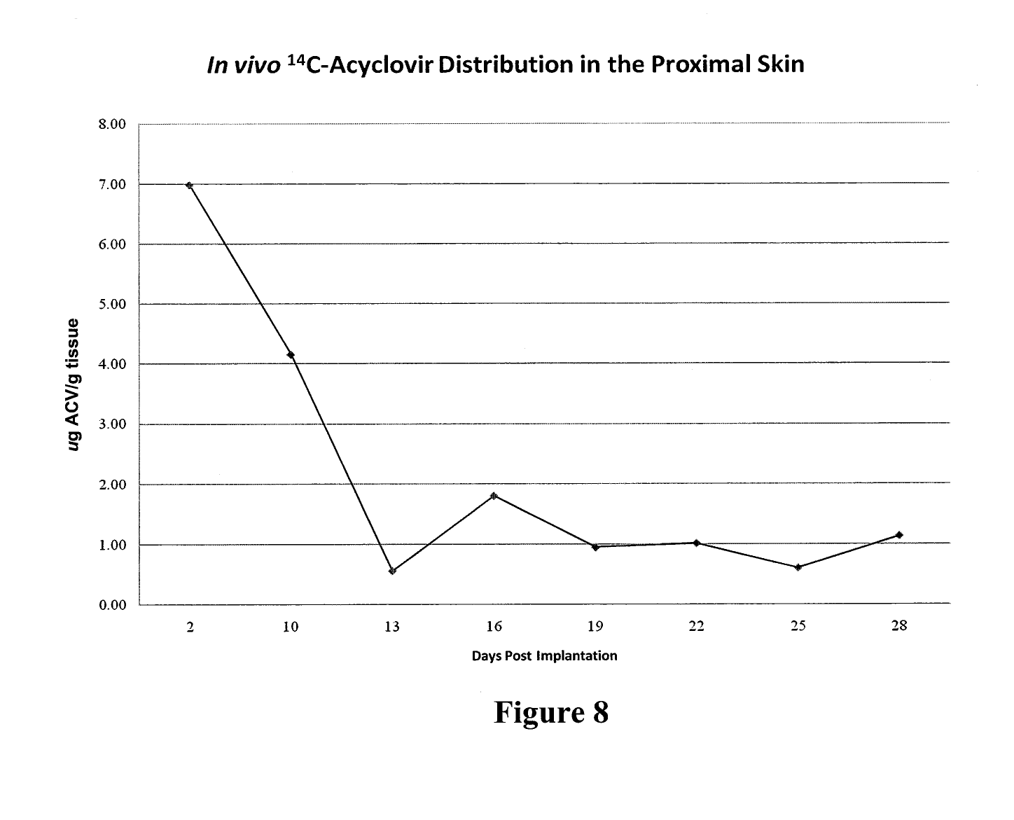

To determine whether the ACV implants exhibited true antiviral activity, mock-infected (FIGS. 8A-C) and HSV-1 (KOS)-infected (FIGS. 8D-F) Vero cells were treated with no ACV (FIGS. 8A & 8D), 0.22 ug of ACV (FIGS. 8B & 8E), or two ACV implants (FIGS. 8C & 8F). All mock-infected Vero cells remained healthy 48 hours after infection (FIGS. 8A-8C). However, HSV-1-infected cells exhibited significant cytopathic effect within 24 hours, including the formation of large plaques within 48 hours (FIG. 8D). Moderate CPE (cell rounding), but no plaque formation, was seen in ACV-treated, HSV-1-infected cells (FIG. 8E), and very little CPE was detected in ACV implant-treated, HSV-1-infected cells, with no plaque formation at 48 hours post-infection (FIG. 8F).

Testing of Implant in Animal Model

Eight-week-old female, hairless, immunocompetent mice, (strain SKH-1) (Charles River Laboratories, Wilmington, Mass.) were infected with 6.3×10⁵ pfu/20 μL of HSV-1 (KOS) through a linear abrasion on the right flank or left uninfected.

After primary infection, the mice were allowed to heal, permitting the virus to establish latency, most likely in the dorsal root ganglia. Mice developed a primary infection that was typical of zosteriform simplex within 10 days. After lesions were completely healed the mice were separated into four treatment groups: 1) no treatment (n=33), 2) two implants composed of only silicone (n=34), 3) two ACV-silicone implants (n=37), and 4) animals subjected to injection with no implant (n=15). Implants were administered subcutaneously using a 12-gauge needle microchip injector (Home Again Pet Recovery Service, East Syracuse, N.Y.), and the wound was closed with surgical glue. Mice were allowed to heal for 10 days after implantation. Mice were then exposed twice to UV light (Foto/Prep I; Fotodyne Incorporated, Hartland, Wis.), 90 seconds per exposure, 24 hours apart. A control group of mice that were not infected with HSV-1 (n=4) were also exposed to the same doses of UV light. Experiments were repeated on a second set of mice to arrive at a larger numbers of subjects, in order to assure reproducibility and all data were compiled into a single group of data as shown below in Table 1.

Both HSV-1-infected and uninfected mice exhibited mild sunburn, with associated flaking of the skin, when exposed to UV light in order to reactivate the latent infection; therefore; it was difficult to determine reactivation based solely on gross observation of lesions alone. To ensure proper classification of a bona fide HSV-1 reactivation event, skin that appeared to be damaged by either sunburn or a reactivation was swabbed to collect any potential HSV-1-infected epithelial cells or HSV-1 virions. The right flank of each mouse was wiped with a Dacron-tipped swab, that was saturated with DMEM culture medium, on days 3, 4, 5, and 6 after UV exposure. Uninfected, UV-exposed mice were also swabbed in the same fashion. The swabs were then agitated in 15 mL conical tubes containing approximately 400 uL of DMEM culture medium to release collected virus. Viral DNA was subsequently isolated from this fluid using a commercial kit (QIAamp DNA Mini Kit; Qiagen, Valencia, Calif.).

Only the swabbings that showed virus presence by PCR were classified as reactivations, regardless of the visual observations made. A minimum of 31 HSV-1 genomes were required in order to be reliably detected in these assays, therefore, if there were a recrudescence with virus or infected-cell quantities below this limit, they would not have been detected and would be deemed negative for reactivation in our assay. HSV-1 was detected via PCR, after a first or second attempt at reactivation, in 18 of the 33 mice in the no treatment group (two separate reactivations; 15 mice were used in only a single reactivation, while 18 mice were used in both reactivations), 27 of the 34 mice that received silicone-only implants (21 only in a single reactivation; 34 in both reactivations), and 12 of the 37 mice that received ACV implants (20 only in a single reactivation; 37 in both reactivations).

TABLE 1
In vivo Reactivation of HSV-1 in Implant-Treated
and Untreated Mice

| | 1st reactivation | 2nd reactivation |
|---|---|---|
| Implant type | (# mice positive) | (# mice positive) |
| None | 11/33 | 7/18 |
| Silicone-only | 19/34 | 8/13 |
| ACV-silicone | 8/37 | 4/17 |
| (Injector only) | 6/15 | N/A |

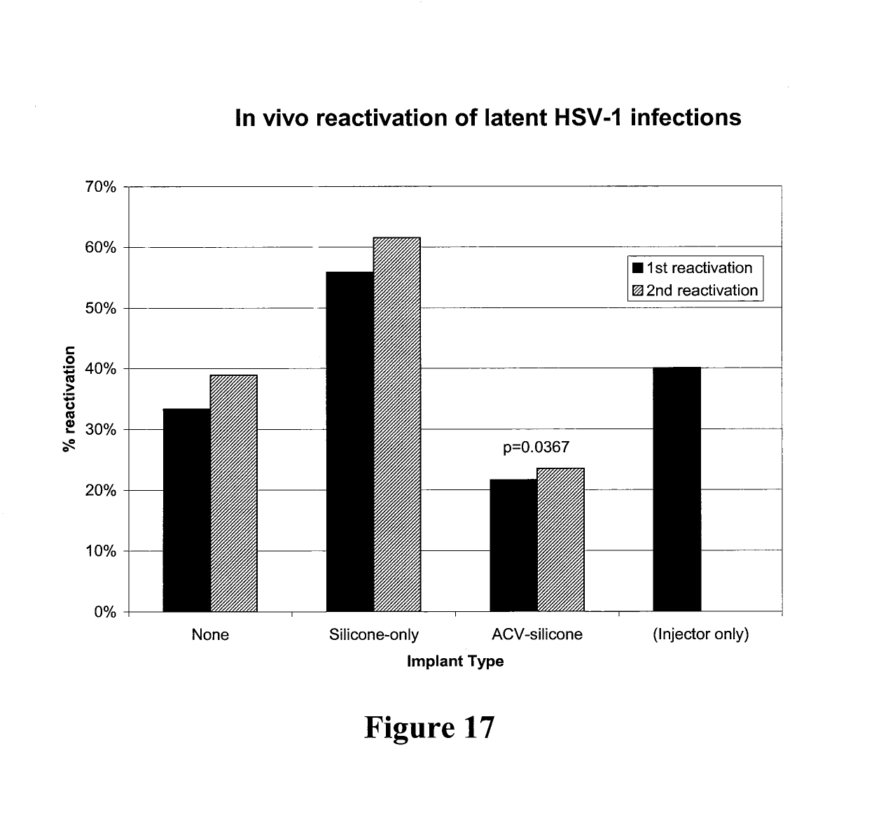

As shown in FIG. 17, there was a significant difference in reactivation levels between the untreated and ACV-implant-treated groups (logistic regression, p=0.0367) and there was no significant difference between the untreated and silicone-only implant groups (logistic regression, p=0.7268). One group of latently HSV-1-infected mice that were subjected to only the trauma of the injection were also analyzed for reactivation. It was found that 6 of those 15 mice were positive for recrudescence, which showed no significant difference (p=0.2621) with the control, untreated group. A second reactivation was not attempted with this group.

Discussion

Herpesviruses are medically important pathogens that cause a diverse range of illnesses. Of interest to the medical community and society as a whole are the development, production, and economic accessibility of antiviral drugs effective against these pathogens. ACV is an excellent antiviral drug that is highly effective and safe; however, the cost associated with this compound is substantial, mainly due to its poor bioavailability; most suppressive therapies utilizing ACV require doses between 800 and 1000 mg daily, costing between $34.92 per month, for generic ACV, to $288.54 per month, for Zovira, GlaxoSmtihKline's trade name form of ACV. However, only 10-20% of the orally dosed drug actually enters the circulation; the majority of the drug is excreted intact (Laskin et al., 1982). Additionally, the 50% inhibitory dose of ACV against HSV-1 has been determined to be about 0.03 ug/mL (Crumpacker et al., 1979; Laskin et al., 1982). Therefore, oral dosing of ACV results in a considerable amount of wasted drug and, hence, wasted money.

The present invention shows that ACV can be incorporated into and released from a silicone-based delivery vehicle. A visually homogeneous mixture of silicone and ACV was produced by simply milling the components together on a two-roll mill press. This visual observation is corroborated by the release kinetics between replicates of the same test group being variable, but not significantly different.

The general trend of release kinetics, in drug released per unit time as a function of the exposed surface area of silicone-containing ACV, followed a logarithmic pattern. ACV was released at much higher rates from the uncovered rod and the uncovered disk implants than from the covered rod or the covered disk. The uncovered disk released amounts of ACV per unit time that were comparable to those of the uncovered rod, even though the former had a smaller solvent-exposed surface area. However, the uncovered disk was not utilized in subsequent research because it was found to be flimsy and easily torn, while the uncovered rod was pliable, but remained intact during the course of normal experimental manipulations.

Neither the covered disk nor the covered rod released appreciable amounts of ACV over the course of the study. Therefore, the uncovered rod, which on average released 0.2-5.0 ug per day, was chosen as the implant style for more complete studies.

The highly variable release kinetics seen from the uncovered rod implants are somewhat common in controlled release devices. The use of manufacturing processes will mitigate variability between individual implants, which in this study were made by hand, and therefore had more variability than would ordinarily be desired.

The implants of the present invention are intended for use in long-term suppressive therapy of HSV-1. It is estimated that a 15-mm long uncovered rod would contain about 10,500 ug of ACV. The initial five-day burst for the uncovered rods released approximately 118 ug of ACV. Even with continuous release of 0.5-2.0 ug of ACV per day (the maximal release rate, see FIGS. 3-6) there is enough drug for the implant to last theoretically about five years.

Next it was of interest to determine where the drug localizes in vivo. Implants were created with [¹⁴C]-acyclovir to trace the location and concentration of drug in vivo in mice. One 15-mm implant was subcutaneously injected lateral to the spinal column, and necropsies were performed over 28 days in 16 mice. Proximal and distal skin, muscle, blood, liver, kidney, spleen, urine, feces, intestine, spinal cord, and brain tissue were isolated and ¹⁴C was detected using a liquid scintillation counter. The skin that was near where the implant was injected showed six times the concentration of drug compared to skin distal to the implant and to the brain. Near zero-order release kinetics of the drug were observed in the proximal skin subsequent to the first ten days of release, consistent with our previous in vitro studies on drug release. The concentration of drug is above the minimum suppressive level in the organs which are affected by HSV-1. It was conclude that location of the implant either proximal to the site of latency or near to the location of clinical symptoms results in localized acyclovir delivery at levels sufficient to suppress reoccurrence of HSV-1 replication over the long term.

Rod shaped implants (2 mm×15 mm) were made by combining silicone (Nusil Med-4050) with ¹⁴C-labeled acyclovir (0.625 μCi/12 mg/ACV/Implant). Two implants were subcutaneously injected lateral to the spinal column in 16 SKH-1 experimental mice. Two experimental mice and one control mouse were euthanized and necropsied at 2, 10, 13, 16, 19, 22, 25, and 28 days post-implantation. During each necropsy, proximal and distal skin samples were taken and homogenized in scintillation fluid. The samples were then incubated at room temperature overnight to allow for diffusion of the ¹⁴C-ACV into the fluid. The radioactivity in each sample was then measured using a liquid scintillation counter.

The local versus systemic ratio was calculated by first determining the [¹⁴C] decays per minute (dpm) per g of tissue for each sample, then subtracting the dpm/g for proximal muscle of the non-treated mouse from the dpm/g for proximal muscle of the treated mouse. The same calculation was performed for distal skin samples, and the ratio of proximal:distal skin dpm/g was employed to demonstrate local v. systemic ACV distribution.

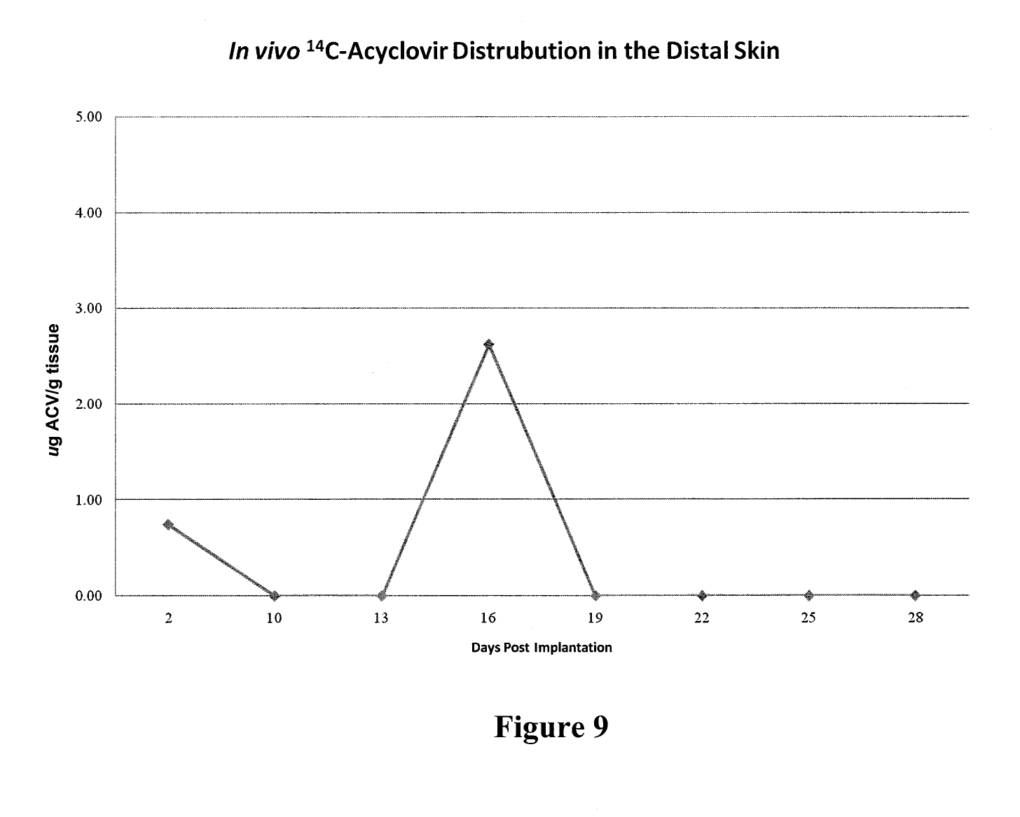

After an initial high release rate from the implant, an average drug release of 1 ug/g/day is observed. This indicates that over this 28 day study, the implant is capable of near zero-order release kinetics. There was also 6× more acyclovir localized in the proximal skin (FIG. 8) versus the distal skin (FIG. 9). This shows that the drug release is primarily local instead of systemic.

Testing in distal skin as shown in FIG. 9 included implants that were constructed exactly as described in the legend to FIG. 8. Mice were implanted and sacrificed, samples were obtained, and [¹⁴C]-ACV was assayed all exactly as described for FIG. 8. The drug levels found in the distal skin to the implant are significantly lower then the levels found in the proximal skin. There is a small amount of drug found during the initial high rate of release in the beginning, as well as a spike at day 16. Similar spikes were found in other tissues at day 16.

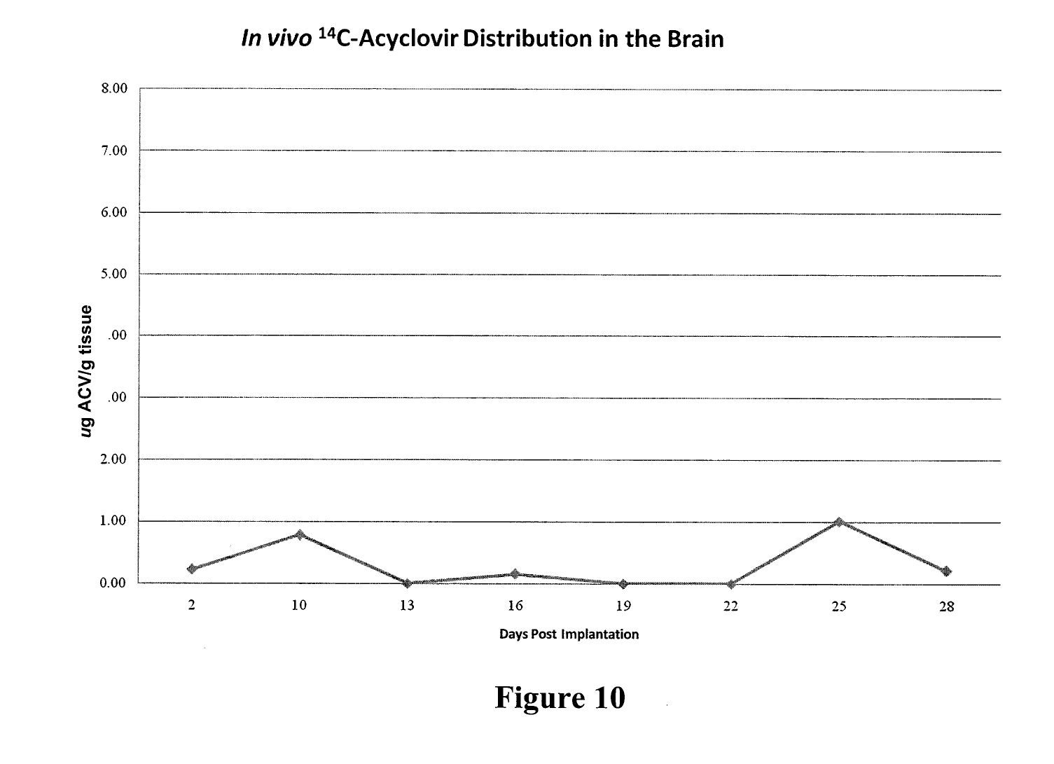

FIG. 10 shows that there was minimal drug found in the brain. The drug may be co-transported under specific physiological conditions, but there is no pattern of sustained uptake of ACV into the central nervous system. Implants were constructed exactly as described in the legend to FIG. 8 and samples were obtained, and [¹⁴C]-ACV was assayed all exactly as described for FIG. 8.

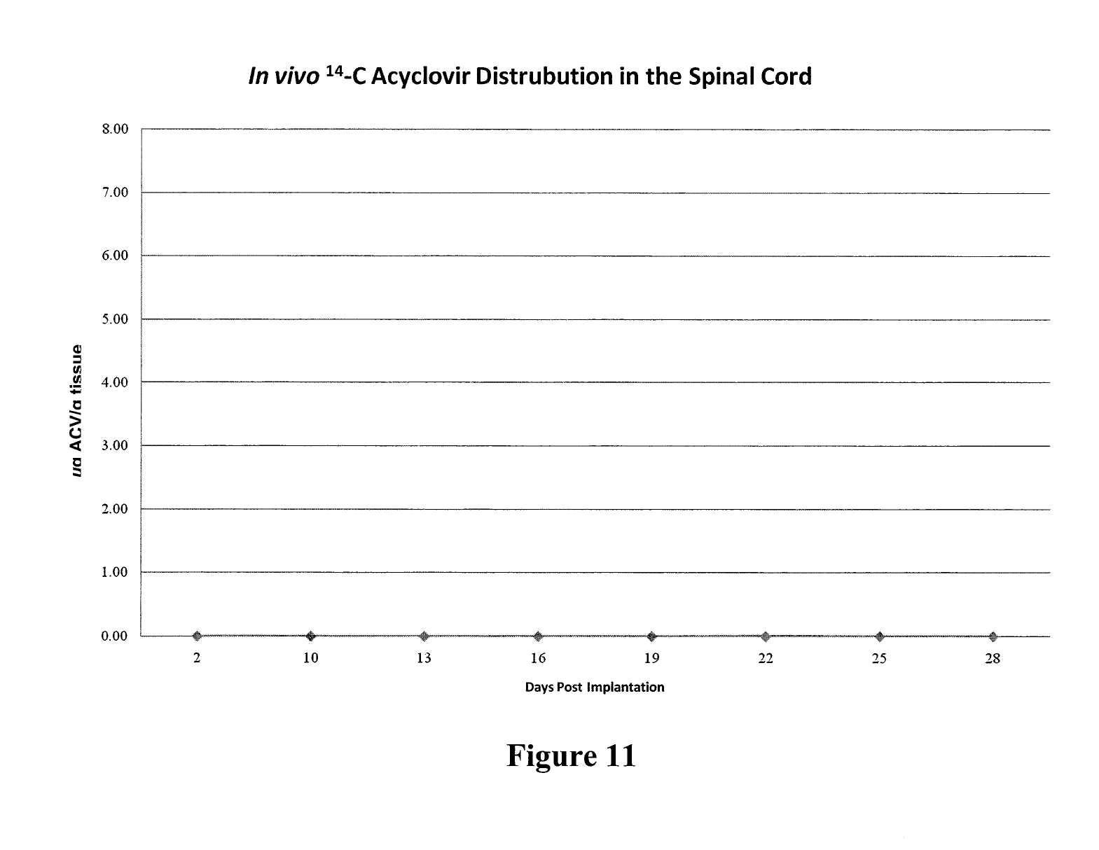

Notably, as shown in FIG. 11, there was no acyclovir found in the spinal cord at any time during this experiment. These results, along with those in FIG. 10, indicate that the drug does not easily cross the blood/brain barrier into the central nervous system.

The results shown in FIGS. 8 and 9 demonstrate effective delivery of locally high concentrations of ACV, at sustained levels more than sufficient to suppress HSV-1 reactivations over the study period.

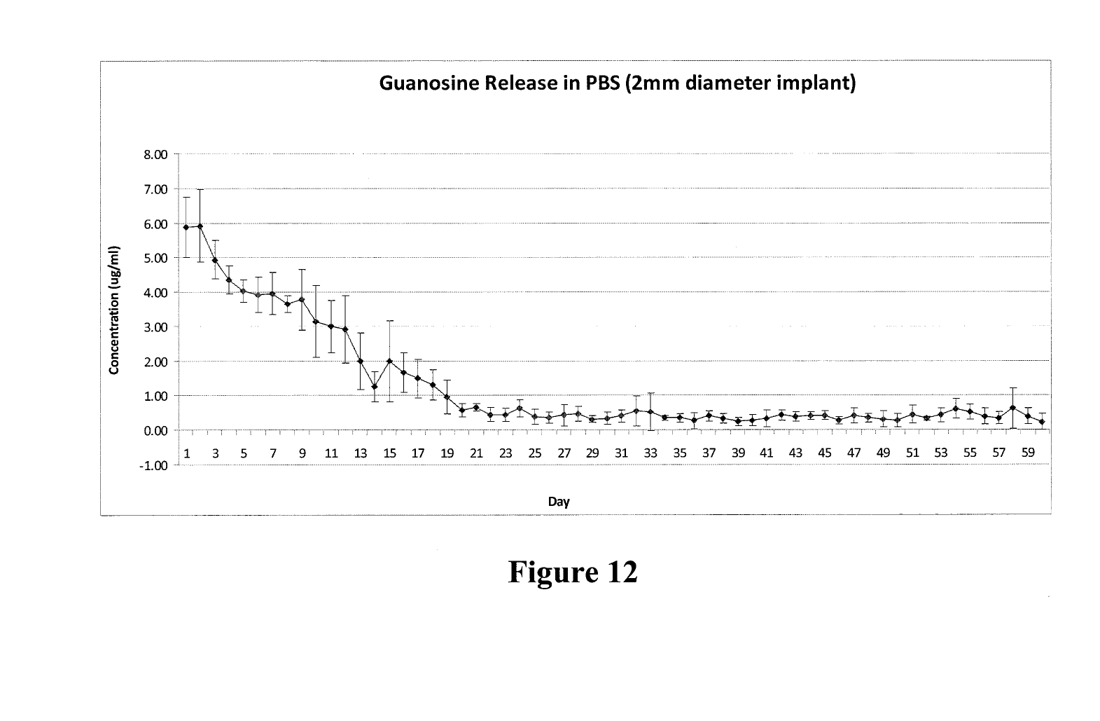

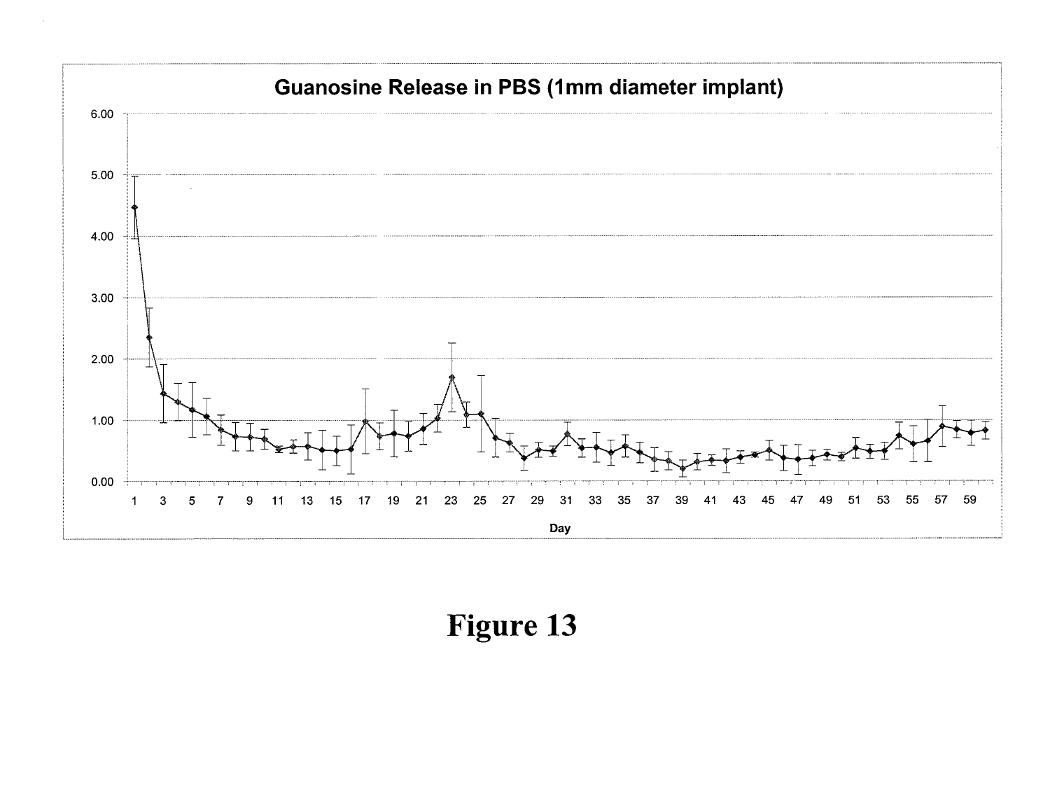

FIGS. 12 to 16 show the results of different types of active ingredients and implants with different diameters. FIG. 12 show the result of using Guanosine which is the ribose-containing counterpart of acyclovir (a.k.a., acycloguanosine). Notably, there was a lower level of chemical released (0.41±0.11 ug/day) from the 2 mm implant relative to the ACV released from 2 mm diameter ACV implants. However the smaller diameter (1 mm) implant releases slightly more guanosine per unit time (0.61±0.26 ug/day) as shown in FIG. 13.

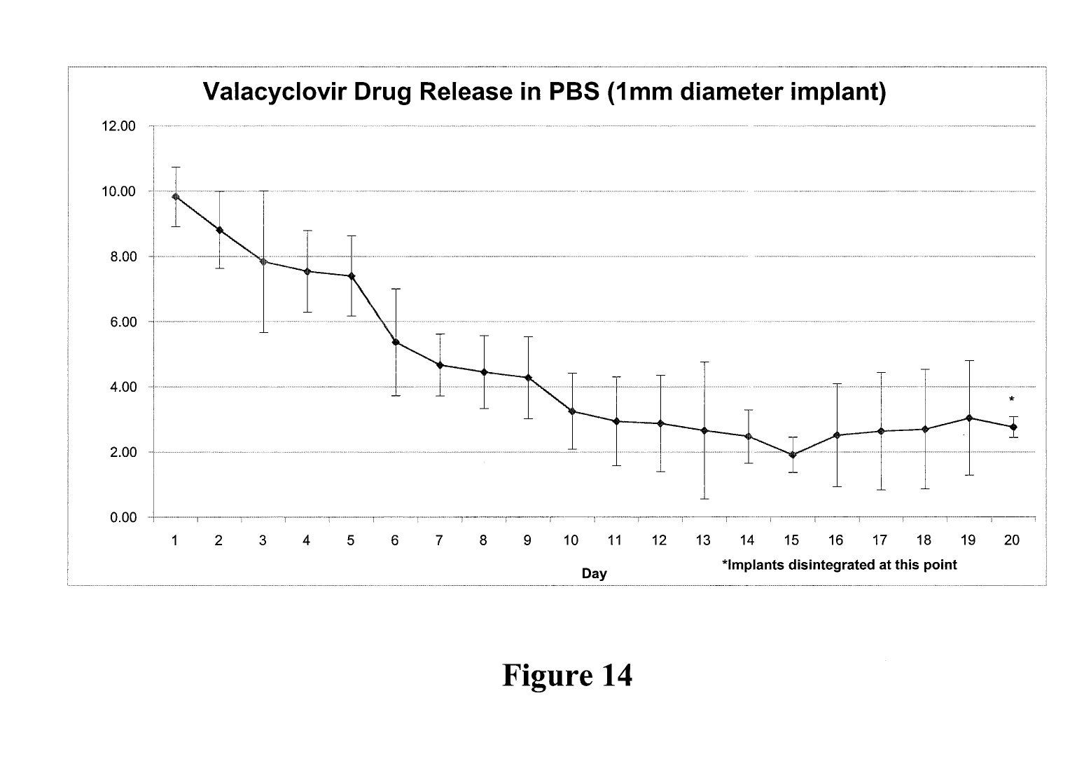

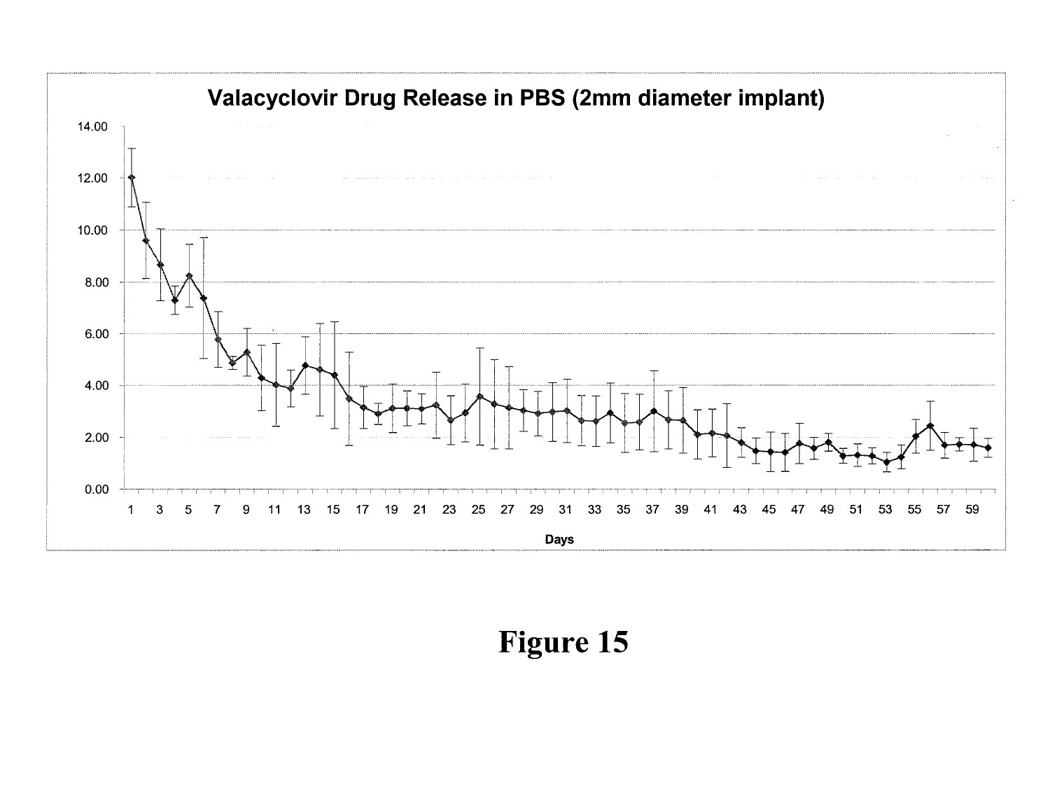

FIG. 14 shows the result using valacyclovir (VCV) which is the valine ester of acyclovir; esterification enhances oral bioavailability of the hydrolyzed ACV. These 1 mm VCV implants showed extremely variable release kinetics between devices (high standard deviation for each measurement) and displayed limited structural integrity, disintegrating into untenable implants within 3 weeks of in vitro study. Nominal drug release was approximately 2.70±0.350 ug/day. As the diameter of the implant increased as shown in FIG. 15, the VCV exhibited higher structural integrity, yet never seemed to release VCV at near zero-order kinetics (note the slope of the line from day 15 appears to be negative, indicating continuously worsening drug release). The marginally flat section of the graph has a daily release of 2.36±0.730 ug/day. Notably, these implants exhibited limited structural integrity.

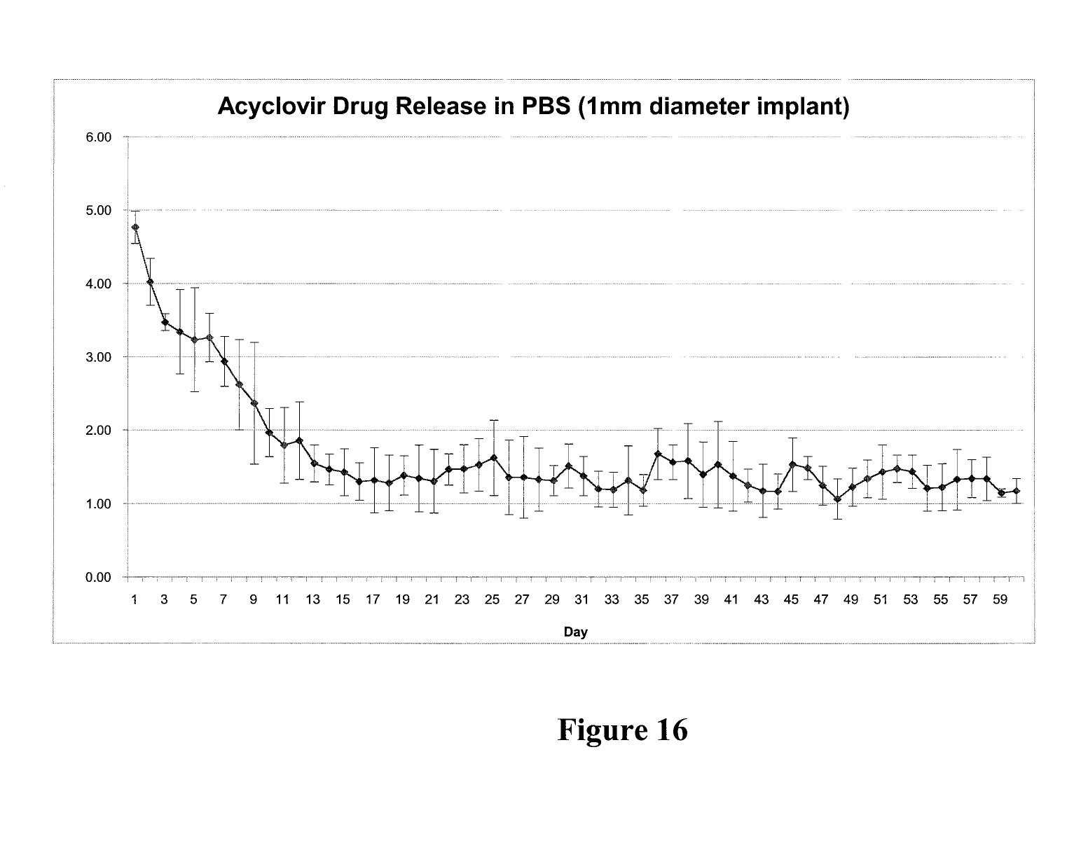

FIG. 16 shows the results of a using a 1 mm ACV implant and the amount of drug release. The amount of drug released per unit time (1.36±0.140 ug/day) is approximately equal to the release rates of 2 mm diameter ACV implants (1.0 to 1.5 ug/day).

The implanted drug delivery device of the present invention was aimed at delivering ACV locally instead of systemically. The advantages of local delivery include: a reduced amount of drug required, and therefore a reduced cost, associated with the treatment. Another advantage of local delivery is a reduction in the side effects associated with systemic treatment, which are nevertheless negligible in the case of ACV (Laskin et al., 1982; Lietman, 1982). Local delivery is not without its drawbacks, however. As seen in the case of Vitrasert, occasionally local delivery cannot completely control the infection, and the use of local delivery may allow for disseminated disease distal to the site of treatment (Musch et al., 1997). However, in the case of HSV-1, spread of the disease beyond the trigeminal ganglia or other nerves that enervate the facial epithelium is unlikely, due to the limited cellular tropism of this virus (Strauss, 2002; Whitley, 2001) and immune control of the initial infection, to prevent its autologous spread in the host (Mikloska and Cunningham, 2001; Mikloska et al., 1999; Schmid and Rouse, 1992). With implants placed in close proximity to both of the paired trigeminal ganglia, complete suppression is possible.

It has been shown that ACV can successfully be incorporated into a silicone-based controlled release device that is capable of delivering functional ACV. Surface area had a significant impact on the release of ACV; however, neither pH nor temperature had an impact on release of ACV from the implant. The ACV implant was successful at protecting Vero cells in culture against CPE caused by HSV-1 and at protecting animals in vivo against HSV-1 reactivation, thereby indicating that the ACV released from the implant was functional and could be delivered at an operationally useful dose.

### REFERENCES

The contents of all references cited herein are hereby incorporated by reference herein for all purposes.
Baringer, J. R. and Swoveland, P. (1973) Recovery of herpes-simplex virus from human trigeminal ganglions. N Engl J Med 288(13), 648-50.
Benedetti, J., Corey, L. and Ashley, R. (1994) Recurrence rates in genital herpes after symptomatic first-episode infection. Ann Intern Med 121(11), 847-54.
Chew, B. H., Knudsen, B. E. and Denstedt, J. D. (2004) The use of stents in contemporary urology. Curr Opin Urol 14(2), 111-5.
Chew, B. H. a. J. D. D. (2004) Technology Insight: novel ureteral stent materials and designs. Nature Clinical Practice Urology 1(1), 44-48.
Corey, L. and Spear, P. G. (1986a) Infections with herpes simplex viruses (1). N Engl J Med 314(11), 686-91.
Corey, L. and Spear, P. G. (1986b) Infections with herpes simplex viruses (2). N Engl J Med 314(12), 749-57.
Corey, L., Wald, A., Patel, R., Sacks, S. L., Tyring, S. K., Warren, T., Douglas, J. M., Jr., Paavonen, J., Morrow, R. A., Beutner, K. R., Stratchounsky, L. S., Mertz, G., Keene, O. N., Watson, H. A., Tait, D. and Vargas-Cortes, M. (2004) Once-daily valacyclovir to reduce the risk of transmission of genital herpes. N Engl J Med 350(1), 11-20.
Crumpacker, C. S., Schnipper, L. E., Zaia, J. A. and Levin, M. J. (1979) Growth inhibition by acycloguanosine of herpesviruses isolated from human infections. Antimicrob Agents Chemother 15(5), 642-5.
de Miranda, P. and Blum, M. R. (1983) Pharmacokinetics of acyclovir after intravenous and oral administration. J Antimicrob Chemother 12 Suppl B, 29-37.
Dobson, C. B., Wozniak, M. A. and Itzhaki, R. F. (2003) Do infectious agents play a role in dementia? Trends Microbiol 11(7), 312-7.
Erlich, K. S., Mills, J., Chatis, P., Mertz, G. J., Busch, D. F., Follansbee, S. E., Grant, R. M. and Crumpacker, C. S. (1989) Acyclovir-resistant herpes simplex virus infections in patients with the acquired immunodeficiency syndrome. N Engl J Med 320(5), 293-6.
Ferguson, T. H., G. F. Needham and J. F. Wagner. (1988) Compudose: An implant system for growth promotion and feed efficiency in cattle. Journal of Controlled Release 8, 45-54.
Furman, P. A., St Clair, M. H., Fyfe, J. A., Rideout, J. L., Keller, P. M. and Elion, G. B. (1979) Inhibition of herpes simplex virus-induced DNA polymerase activity and viral DNA replication by 9-(2-hydroxyethoxymethyl)guanine and its triphosphate. J Virol 32(1), 72-7.
Furman, P. A., St Clair, M. H. and Spector, T. (1984) Acyclovir triphosphate is a suicide inactivator of the herpes simplex virus DNA polymerase. J Biol Chem 259(15), 9575-9.
Fyfe, J. A., Keller, P. M., Furman, P. A., Miller, R. L. and Elion, G. B. (1978) Thymidine kinase ftom herpes simplex virus phosphorylates the new antiviral compound, 9-(2-hydroxyethoxymethyl)guanine. J Biol Chem 253(24), 8721-7.
Gupta, R., Wald, A., Krantz, E., Selke, S., Warren, T., Vargas-Cortes, M., Miller, G. and Corey, L. (2004) Valacyclovir and acyclovir for suppression of shedding of herpes simplex virus in the genital tract. J Infect Dis 190(8), 1374-81.
Hirsch, M. A. (1995) Herpes Simplex Virus. In: J. E. B. G. L. Mandell, Dolin (Ed), Mandell, Douglas and Bennett's Principles and Practice of Infectious Diseases, 6th ed., pp. 1336-1345. Churchill Livingstone, New York, N.Y.
Hook, E. W., 3rd, Cannon, R. O., Nahmias, A. J., Lee, F. F., Campbell, C. H., Jr., Glasser, D. and Quinn, T, C. (1992) Herpes simplex virus infection as a risk factor for human immunodeficiency virus infection in heterosexuals. J Infect Dis 165(2), 251-5.
Itzhaki, R. F., Lin, W. R., Shang, D., Wilcock, G. K., Faragher, B. and Jamieson, G. A. (1997) Herpes simplex virus type 1 in brain and risk of Alzheimer's disease. Lancet 349(9047), 241-4.
Jamieson, G. A., Maitland, N. J., Wilcock, G. K., Craske, J. and Itzhaki, R. F. (1991) Latent herpes simplex virus type 1 in normal and Alzheimer's disease brains. J Med Virol 33(4), 224-7.
Kajihara, M., Sugie, T., Hojo, T., Maeda, H., Sano, A., Fujioka, K., Sugawara, S. and Urabe, Y. (2001) Development of a new drug delivery system for protein drugs using silicone (II). J Control Release 73(2-3), 279-91.
Kajihara, M., Sugie, T., Maeda, H., Sano, A., Fujioka, K., Urabe, Y., Tanihara, M. and Imanishi, Y. (2003) Novel drug delivery device using silicone: controlled release of insoluble drugs or two kinds of water-soluble drugs. Chem Pharm Bull (Tokyo) 51(1), 15-9.
Kajihara, M., Sugie, T., Mizuno, M., Tamura, N., Sano, A., Fujioka, K., Kashiwazaki, Y., Yamaoka, T., Sugawara, S. and Urabe, Y. (2000) Development of new drug delivery system for protein drugs using silicone (J). J Control Release 66(1), 49-61.
Khanavkar, B. and Ewig, S. (2004) A new tool for an old disease. Eur Respir J 24(3), 343-4.
Laskin, O. L., Longstreth, J. A., Saral, R., de Miranda, P., Keeney, R. and Lietman, P. S. (1982) Pharmacokinetics and tolerance of acyclovir, a new anti-herpesvirus agent, in humans. Antimicrob Agents Chemother 21(3), 393-8.
Lietman, P. S. (1982) Acyclovir clinical pharmacology. An overview. Am J Med 73(1A), 193-6. Maeda, H., Ohashi, E., Sano, A., Kawasaki, H. and Kurosaki, Y. (2003) Investigation of the release behavior of a covered-rod-type formulation using silicone. J Control Release 90(1), 59-70.
Maeda, H., Sugie, T., Sano, A., Kawasaki, H. and Kurosaki, Y. (2004) Study on accelerated evaluation system for release profiles of covered-rod type silicone formulation using indomethacin as a model drug. J Control Release 94(2-3), 337-49.
Maeda, M., Moriuchi, S., Sano, A. and Yoshimine, T. (2002) New drug delivery system for water-soluble drugs using silicone and its usefulness for local treatment: application of GCV-silicone to GCV/HSV-tk gene therapy for brain tumor. J Control Release 84(1-2), 15-25.
Martin, J. L., Brown, C. E., Matthews-Davis, N. and Reardon, J. E. (1994) Effects of antiviral nucleoside analogs on human DNA polymerases and mitochondrial DNA synthesis. Antimicrob Agents Chemother 38(12), 2743-9.
Mikloska, Z. and Cunningham, A. L. (2001) Alpha and gamma interferons inhibit herpes simplex virus type 1 infection and spread in epidermal cells after axonal transmission. J Virol 75(23), 11821-6.
Mikloska, Z., Sanna, P. P. and Cunningham, A. L. (1999) Neutralizing antibodies inhibit axonal spread of herpes simplex virus type 1 to epidermal cells in vitro. J Virol 73(7), 5934-44.
Miller, C. S., Avdiushko, S. A., Kryscio, R. J., Danaher, R. J. and Jacob, R. J. (2005) Effect of prophylactic valacyclovir on the presence of human herpesvirus DNA in saliva of healthy individuals after dental treatment. J Clin Microbiol 43(5), 2173-80.
Miller, W. H. and Miller, R. L. (1982) Phosphorylation of acyclovir diphosphate by cellular enzymes. Biochem Pharmacol 31(23), 3879-84.
Musch, D. C., Martin, D. F., Gordon, J. F., Davis, M. D. and Kuppermann, B. D. (1997) Treatment of cytomegalovirus retinitis with a sustained-release ganciclovir implant. The Ganciclovir Implant Study Group. N Engl J Med 337(2), 83-90.
Nash, H. A., Robertson, D. N., Moo Young, A. J. and Atkinson, L. E. (1978) Steroid release from silastic capsules and rods. Contraception 18(4), 367-94.
Park, H. and Park, K. (1996) Biocompatibility issues of implantable drug delivery systems. Pharm Res 13(12), 1770-6.
Reitano, M., Tyring, S., Lang, W., Thoming, C., Worm, A. M., Borelli, S., Chambers, L. O., Robinson, J. M. and Corey, L. (1998) Valaciclovir for the suppression of recurrent genital herpes simplex virus infection: a large-scale dose range-finding study. International Valaciclovir HSV Study Group. J Infect Dis 178(3), 603-10.
Robertson, D. N., Sivin, I., Nash, H. A., Braun, J. and Dinh, J. (1983) Release rates of levonorgestrel from Silastic capsules, homogeneous rods and covered rods in humans. Contraception 27(5), 483-95.
Saral, R., Burns, W. H., Laskin, O. L., Santos, G. W. and Lietman, P. S. (1981) Acyclovir prophylaxis of herpes-simplex-virus infections. N Engl J Med 305(2), 63-7.
Schmid, D. S. and Rouse, B. T. (1992) The role of T cell immunity in control of herpes simplex virus. Curr Top Microbiol Immunol 179, 57-74.
Shier, D. a. J. B., Ricki Lewis. (2004) Hole's human anatomy and physiology. 10th ed. McGraw-Hill, New York, N.Y.
Sivin, I., Lahteenmaki, P., Ranta, S., Damey, P., Klaisle, C., Wan, L., Mishell, D. R., Jr., Lacarra, M., Viegas, O. A., Bilhareus, P., Koetsawang, S., Piya-Anant, M., Diaz, S., Pavez, M., Alvarez, F., Brache, V., LaGuardia, K., Nash, H. and Stem, J. (1997) Levonorgestrel concentrations during use of levonorgestrel rod (LNG ROD) implants. Contraception 55(2), 81-5.
Smith, S. R., Butterly, D. W., Alexander, B. D. and Greenberg, A. (2001) Viral infections after renal transplantation. Am J Kidney Dis 37(4), 659-76.
Spruance, S. L., Overall, J. C., Jr., Kern, E. R., Krueger, G. G., Pliam, V. and Miller, W. (1977) The natural history of recurrent herpes simplex labialis: implications for antiviral therapy. N Engl J Med 297(2), 69-75.
Strauss, J. H., E. G. Strauss. (2002) Viruses and Human Disease. Academic Press, San Diego, Calif.
Tang, Y. W., Mitchell, P. S., Espy, M. J., Smith, T. F. and Persing, D. H. (1999) Molecular diagnosis of herpes simplex virus infections in the central nervous system. J Clin Microbiol 37(7), 2127-36.
Theil, D., Arbusow, V., Derfuss, T., Strupp, M., Pfeiffer, M., Mascolo, A. and Brandt, T. (2001) Prevalence of HSV-1 LAT in human trigeminal, geniculate, and vestibular ganglia and its implication for cranial nerve syndromes. Brain Pathol 11 (4), 408-13.
Tiffany, J. S., D. J. Petraitis. (1995) Silicone biomaterials. In: D. L. Wise (Ed), Encyclopedic handbook of biomaterials and bioengineering. Vol. 2. Marcel Dekker, New York.
Tolle-Sander, S., Lentz, K. A., Maeda, D. Y., Coop, A. and Polli, J. E. (2004) Increased acyclovir oral bioavailability via a bile acid conjugate. Mol Pharm 1(1), 40-8.
Tyring, S. K., Baker, D. and Snowden, W. (2002) Valacyclovir for herpes simplex virus infection: long-term safety and sustained efficacy after 20 years' experience with acyclovir. J Infect Dis 186 Suppl 1, S40-6.
Wald, A. and Link, K. (2002) Risk of human immunodeficiency virus infection in herpes simplex virus type 2-seropositive persons: a meta-analysis. J Infect Dis 185(1), 45-52.
Wald, A., Zeh, J., Barnum, G., Davis, L. G. and Corey, L. (1996) Suppression of subclinical shedding of herpes simplex virus type 2 with acyclovir. Ann Intern Med 124(1 Pt 1), 8-15.
Weller, S., Blum, M. R., Doucette, M., Burnette, T., Cederberg, D. M., de Miranda, P. and Smiley, M. L. (1993) Pharmacokinetics of the acyclovir pro-drug valaciclovir after escalating single- and multiple-dose administration to normal volunteers. Clin Pharmacol Ther 54(6), 595-605.
Whitley, R. J. (2001) Herpes Simplex Virus. In: D. M. Knipe, P. M. Howley (Ed), Fields Virology, 4th ed., pp. 2461-2511. Vol. 2. 2 vols. Lippincott, Williams, and Wilkins, Philadelphia, Pa.
Whitley, R. J. and Roizman, B. (2001) Herpes simplex virus infections. Lancet 357(9267), 1513-8.

That which is claimed is:

1. A drug delivery device comprising:
a substrate having a geometric shape for implantation within a subject, wherein the substrate comprises a polymeric or polymeric containing material and at least one therapeutic compound, and wherein the therapeutic compound is dissolved, dispersed or impregnated into at least the outer surface of the substrate.

2. The drug delivery device of claim 1, wherein the geometric shape is selected from the group consisting of a rod, bundle of multiple rods, disk, doughnut, helical, elliptical, triangular and oval.

3. The drug delivery device of claim 2, wherein the therapeutic compound is acyclovir or valacyclovir or functional analogue thereof.

4. The drug delivery device of claim 3, wherein the therapeutic compound is in an amount to deliver from about 0.02 to 50 ug/day.

5. The drug delivery device of claim 3, wherein the therapeutic compound is in an amount to deliver from about 0.5 to 2 ug/day.

6. The drug delivery device of claim 3, wherein the rod has a length of approximately 5 mm to 15 mm.

7. The drug delivery device of claim 1, wherein the substrate comprises a homogeneous mixture of the therapeutic compound and polymeric material.

8. The drug delivery device of claim 1, wherein implantation within the subject is at or near the site of latent infection or at the site of observed clinical symptoms.

9. The drug delivery device of claim 1, wherein the subject is infected with an alphaherpes virus.

10. The drug delivery device of claim 1, wherein the subject is infected HSV-1, HSV 2 or VZV.

11. The drug delivery device of claim 1, wherein the therapeutic compound diffuses into surrounding biological environment in a controlled released manner.

12. The drug delivery device of claim 1, wherein the therapeutic compound is dispersed throughout the entire substrate.

13. The drug delivery device of claim 6, wherein the polymeric or a polymeric containing material has sufficient flexibility for comfortable inclusion in a subject and sufficient rigidity for ease of insertion.

14. The drug delivery device of claim 1, wherein the substrate is fabricated of multiple layers wherein each layer comprises different concentrations of the therapeutic compound.

15. The drug delivery device of claim 1, wherein the polymeric or polymeric containing material is a biodegradable or a non biodegradable material.

16. A method for treating or controlling a herpesvirus, the method comprising:
implanting into a subject having the need for such treatment, an implantable drug delivery device comprising:
a substrate having a geometric shape for implantation within a subject, wherein the substrate comprises a polymeric or polymeric containing material and at least one therapeutic compound, wherein the therapeutic compound is dissolved, dispersed or impregnated into at least the outer surface of the substrate.

17. The method of claim 16, further comprising implanting the drug delivery device at or near the site of latent infection or at the site of observed clinical symptoms.
trigeminal ganglia, sacral, dorsal root ganglia or other nerves that enervate the facial epithelium.

18. The method of claim 16, wherein the geometric shape is selected from the group consisting of a rod, bundle of multiple rods, disk, doughnut, helical, elliptical, triangular and oval.

19. The method of claim 16, wherein the therapeutic compound is acyclovir, valacyclovir or a functional analogue.

20. The method of claim 19, wherein the therapeutic compound is in an amount to deliver from about 0.02 to 50 ug/day.

21. The method of claim 19, wherein the therapeutic compound is in an amount to deliver from about 0.5 to 2 ug/day.

22. The method of claim 18, wherein the rod has a length of approximately 5 mm to 15 mm.

23. The method of claim 16, wherein the substrate comprises a homogeneous mixture of the therapeutic compound and polymeric material.

24. The method of claim 16, wherein implantation within the subject is at or near the site of latent infection or at the site of observed clinical symptoms.

25. The method of claim 16, wherein the subject is infected with HSV-1, HSV 2 or VZV.

26. The method of claim 16, wherein the therapeutic compound diffuses into surrounding biological environment in a controlled released manner.

27. The method of claim 16, wherein the therapeutic compound is dispersed throughout the entire substrate.

28. The method of claim 16, wherein the polymeric or a polymeric containing material has sufficient flexibility for comfortable inclusion in a subject and sufficient rigidity for ease of insertion.

29. The method of claim 16, wherein the substrate is fabricated of multiple layers wherein each layer comprises different concentrations of the therapeutic compound.

30. The method of claim 16, wherein the polymeric or polymeric containing material is biodegradable or non biodegradable material.

31. A method of fabricating an implantable drug delivery device for implanting in a subject and delivering of a therapeutic compound comprising the steps of:
mixing particles or a solution containing the therapeutic compound with a polymeric material or polymeric containing material and blending until homogeneous; and
forming the substrate by extruding the polymeric combination into the desired geometric shape.

32. A method for treating and controlling HSV-1, the method comprising:
implanting subcutaneously a silicone or silicone-based substrate, wherein the substrate comprises a therapeutic compound for the treatment of HSV, wherein the therapeutic compound is ACV and released from the substrate with near-zero order kinetics.
